(19) United States
(12) Patent Application Publication
(10) Pub. No.: US 2016/0106110 A1
(43) Pub. Date: Apr. 21, 2016

(54) COMPOSITIONS AND METHODS FOR ENHANCING GERMINATION

(71) Applicants: CORNELL UNIVERSITY, Ithaca, NY (US); NOVOZYMES BIOAG A/S, Bagsvaerd (DK)

(72) Inventors: Michael Frodyma, Salem, VA (US); Julia M. Crane, Silver Spring, MD (US); Gary C. Bergstrom, Ithaca, NY (US)

(73) Assignees: NOVOZYMES BIOAG A/S, Bagsvaerd (DK); CORNELL UNIVERSITY, Ithaca, NY (US)

(21) Appl. No.: 14/894,673

(22) PCT Filed: May 23, 2014
(86) PCT No.: PCT/US14/39298

§ 371 Date: Nov. 30, 2015

Related U.S. Application Data
(60) Provisional application No. 61/829,369, filed on May 31, 2013.

(51) Int. Cl. A01N 63/00; C12N 1/20; C12R 1/07

(52) U.S. Cl. CPC A01N 63/00; C12R 1/07; C12N 1/20

(57) ABSTRACT

Described herein are compositions comprising one or more microorganisms and one or more germinants. Further described herein are methods for treating plants, plant parts, soils, with one or more microorganisms and one or more germinants, and compositions thereof.

### FIELD

Compositions comprising one or more microorganisms and one or more germinants as well as methods of treating plants or plant parts with one or more microorganisms and one or more germinants.

### BACKGROUND

Plant growth depends at least in part on interactions between the plant and microorganisms that inhabit the surrounding soil. Biological solutions continue to be explored to enhance plant growth, health and vigor; and these solutions typically take advantage of some biological relationship between the plant and a microorganism. Biological solutions have been used, among other things, to promote plant growth, combat plant pathogens, reduce the use of chemicals for soil fertilization and pest management, and to increase nutrient availability and uptake to the plant. Although some of these chemicals are known to have negative environmental and human health problems, nevertheless such chemical agents continue to be in wide use due to their strong activity against important fungal diseases, and limited availability of environmentally safer and effective alternatives.

Generally, biological solutions are preferred over more traditional synthetic chemical control methodologies as biological solutions usually cause little or no injury to the plant host or the environment, and some may even favor normal plant development. However, biological solutions can sometimes be limited either in the scope of their effectiveness in the remedy which they are to impart or in their ability to survive under practical field conditions and during treatment applications.

Attempts at using biological solutions have been made to control plant fungal diseases by using certain microorganisms. In particular, species of the bacteria Bacillus has been used for this purpose.

For example, U.S. Pat. No. 5,589,381 describes a Bacillus licheniformis strain PR1-36a with some ability to inhibit certain plant pathogens.

U.S. Pat. No. 6,060,051 describes an antibiotic-producing and metabolite-producing Bacillus subtilis strain that exhibits insecticidal, antifungal and antibacterial activity. Also described are methods of protecting or treating plants from fungal and bacterial infections and corn rootworm infestations comprising the step of applying to the plant an effective amount of the antibiotic/metabolite-producing Bacillus subtilis strain, the antibiotic/metabolite produced by the novel Bacillus subtilis strain or a combination thereof.

Notwithstanding, efforts to apply certain live biological organisms have been greatly limited by the insufficient ability of these strains to germinate on minimal carbon source substrates (e.g., a plant leaf following foliar application of a biological control organism). By remaining in the dormant spore state, such organisms are unable to perform thier beneficial modes of action. Germination of such spores would enable the organisms to regain metabolic activity, and thereby, increase the effectiveness of these biological solutions. Therefore, an environmentally safe and effective solution to enhancing the effectiveness of biological solutions remains a long felt need in the agricultural industry over currently used hazardous chemicals.

### SUMMARY

Described herein are compositions comprising one or more microorganisms and one or more germinants as well as methods comprising application of those compositions to promote faster germination of the one or more microorganisms on a substrate not optimal for microbial growth; particularly foliar applications.

In one embodiment, the compositions described herein comprise a carrier, one or more bacteria, and one or more germinants. In a particular aspect, the one or more bacteria are in the spore form. The one or more germinants can include any substance which is capable of inducing the germination of a microbial spore.

In another embodiment, the composition further comprises one or more agriculturally beneficial ingredients, such as one or more biologically active ingredients, one or more micronutrients, one or more biostimulants, one or more preservatives, one or more polymers, one or more wetting agents, one or more surfactants, one or more herbicides, one or more fungicides, one or more insecticides, one or more fertilizers, or combinations thereof.

In one embodiment, the composition described herein further comprises one or more biologically active ingredients. Biologically active ingredients may include one or more plant signal molecules. In a specific embodiment, the one or more biologically active ingredients may include one or more lipo-chitooligosaccharides (LCOs), one or more chitooligosaccharides (COs), one or more chitinous compounds, one or more nod gene inducers (e.g., flavonoid and non-flavonoid nod gene inducers) and derivatives thereof, one or more karrikins and derivatives thereof, or any signal molecule combination thereof

Further described herein is a method of applying to a plant or plant part one or more microorganisms comprising contacting a plant or plant part with one or more microorganisms and one or more germinants. The one or more microorganisms may include bacteria having one or more plant growth promoting properties. The one or more microorganisms may be applied either simultaneously or sequentially, with the one or more germinants.

The method may further comprise subjecting the plant or plant part to one or more agriculturally beneficial ingredients, applied either simultaneously or sequentially, with the one or more microorganisms or one or more germinants. The one or more agriculturally beneficial ingredients can include one or more biologically active ingredients, one or more micronutrients, one or more biostimulants, or combinations thereof. In one embodiment, the method further comprises subjecting the plant or plant part to one or more biologically active ingredients. Biologically active ingredients may one or more plant signal molecules. In a specific embodiment, the one or more biologically active ingredients may include one or more LCOs, one or more chitinous compounds, one or more COs, one or more nod gene inducers (e.g., flavonoid and non-flavonoid nod gene inducers) and derivatives thereof, one or more karrikins and derivatives thereof, or any signal molecule combination thereof.

In a specific embodiment described herein, is a method for inducing the germination of a microorganism comprising foliarly applying one or more microbial spores and one or more germinants to a plant or plant part, wherein upon foliar application of the one or more microbial spores and one or more germinants to a plant or plant part, the one or more microbial spores exhibit increased germination on the plant or plant part compared to the foliar application of microbial spores alone (i.e., without one or more germinants) to a plant or plant part. In a more particular embodiment, the method comprises applying one or more bacterial spores and one or more germinants to plant foliage. The method may further comprise subjecting the plant or plant part to one or more agriculturally beneficial ingredients, applied simultaneously or sequentially with the one or more bacterial spores or one or more germinants.

In still another embodiment is a method for coating seeds with the compositions described herein.

In still yet another embodiment is a method for treating a soil with one or more of the compositions described herein.

### DETAILED DESCRIPTION

The disclosed embodiments relate to compositions and methods for enhancing plant growth.

### DEFINITIONS

As used herein, the singular forms “a”, “an” and “the” are intended to include the plural forms as well, unless the context clearly indicates otherwise.

As used herein, the term “acaricide(s)” means any agent or combination of agents capable of being toxic to an acarid, controlling an acarid, killing an acarid, inhibiting the growth of an acarid, and/or inhibiting the reproduction of an acarid.

As used herein, the term “agriculturally beneficial ingredient(s)” means any agent or combination of agents capable of causing or providing a beneficial and/or useful effect in agriculture.

As used herein, the term “beneficial microorganism(s)”, “beneficial microbe”, “beneficial bacteria”, etc., means any microorganism (e.g., bacteria, fungus, etc., or combinations thereof) having one or more beneficial properties (e.g., produce one or more of the plant signal molecules described herein, enhance nutrient and water uptake, promote and/or enhance nitrogen fixation, enhance growth, enhance seed germination, enhance seedling emergence, increased seed number or size, break the dormancy or quiescence of a plant, etc.).

As used herein, “biologically active agent(s)” means any biological organism or chemical element, molecule, or compound, or mixture thereof, which has a biological activity in a seed, a plant, or a plant part (e.g., plant signal molecules, other microorganisms, gluconolactones, glutathiones, etc.). Non-limiting examples of “biological activity” include N₂ fixation, phosphate solubilization, plant growth-enhancement, bio-pesticidal activity, bio-fungicidal activity, etc.

As used herein, the term “biostimulant(s)” means any agent or combination of agents capable of enhancing metabolic or physiological processes within plants and soils.

As used herein, the term “carrier” means an “agronomically acceptable carrier.” An “agronomically acceptable carrier” means any material which can be used to deliver the actives (e.g., microorganisms described herein, germinants, agriculturally beneficial ingredient(s), biologically active ingredient(s), etc.) to a plant or a plant part (e.g., plant foliage), and preferably which carrier can be applied (to the plant, plant part (e.g., foliage, seed), or soil) without having an adverse effect on plant growth, soil structure, soil drainage or the like.

As used herein, the term “soil-compatible carrier” means any material which can be added to a soil without causing/having an adverse effect on plant growth, soil structure, soil drainage, or the like.

As used herein, the term “seed-compatible carrier” means any material which can be added to a seed without causing/having an adverse effect on the seed, the plant that grows from the seed, seed germination, or the like.

As used herein, the term “foliar-compatible carrier” means any material which can be added to a plant or plant part without causing/having an adverse effect on the plant, plant part, plant growth, plant health, or the like.

As used herein, the terms “effective amount”, “effective concentration”, or “effective dosage” means the amount, concentration, or dosage of the one or more germinants sufficient to induce germination of the one or more microorganisms. The actual effective dosage in absolute value depends on factors including, but not limited to, synergistic or antagonistic interactions between the other active or inert ingredients which may enhance or reduce the germinating effects of the one or more germinatnts, and the stability of the one or more germinants in compositions and/or as plant or plant part treatments. The “effective amount”, “effective concentration”, or “effective dosage” of the one or more germinants may be determined, e.g., by a routine dose response experiment.

As used herein, terms “enhanced plant growth”, “increased plant growth”, “plant growth-enhancement”, or “plant growth-enhancing”, which may all be used interchangeably, means increased plant yield (e.g., increased biomass, increased fruit number, increased bolls, increased seed number or size, or a combination thereof as measured by bushels per acre), increased root number, increased root mass, increased root volume, increased leaf area, increased plant stand, increased plant vigor, faster seedling emergence (i.e., enhanced emergence), faster germination, (i.e., enhanced germination), or combinations thereof.

As used herein, the term “foliage” means all parts and organs of plants above the ground. Non-limiting examples include leaves, needles, stalks, stems, flowers, fruit bodies, fruits, etc. As used herein, the term “foliar application”, “foliarly applied”, and variations thereof, is intended to include application of an active ingredient to the foliage or above ground portions of the plant, (e.g., the leaves of the plant). Application may be effected by any means known in the art (e.g., spraying the active ingredient).

As used herein, the term “fungicide(s)” means any agent or combination of agents capable of being toxic to a fungus, controlling a fungus, killing a fungus, inhibiting the growth of a fungus, and/or inhibiting the reproduction of a fungus.

As used herein, the term “germinant(s)” means any substance or compound that induces microbial spore germination (e.g., a substance or compound that induces the germination of a microbial spore, such as a bacterial spore).

As used herein, the term “herbicide(s)” means any agent or combination of agents capable of being toxic to a weed, controlling a weed, killing a weed, inhibiting the growth of a weed, and/or inhibiting the reproduction of a weed

As used herein, the term “inoculum” means any form of microbial cells, or spores, which are capable of propagating on or in the soil when the conditions of temperature, moisture, etc., are favorable for microbial growth.

As used herein, the term “insecticide(s)” means any agent or combination of agents capable of being toxic to an insect, controlling an insect, killing an insect, inhibiting the growth of an insect, and/or inhibiting the reproduction of an insect.

As used herein, the term “isomer(s)” means all stereoisomers of the compounds and/or molecules referred to herein (e.g., flavonoids, LCOs, COs, chitinous compounds, jasmonic acid or derivatives thereof, linoleic acid or derivatives thereof, linolenic acid or derivatives thereof, kerrikins, amino acids or derivatives thereof, sugars or derivatives thereof, germinants described herein or derivatives thereof, etc.), including enantiomers, diastereomers, as well as all conformers, roatmers, and tautomers, unless otherwise indicated. The compounds and/or molecules disclosed herein include all enantiomers in either substantially pure levorotatory or dextrorotatory form, or in a racemic mixture, or in any ratio of enantiomers. Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose a (S)-enantiomer, that embodiment also includes the (R)-enantiomer; where embodiments disclose a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers, and tautomers of compounds and/or molecules depicted.

As used herein, the term “nematicide(s)” means any agent or combination of agents capable of being toxic to a nematode, controlling a nematode, killing a nematode, inhibiting the growth of a nematode, and/or inhibiting the reproduction of a nematode.

As used herein, the term “nitrogen fixing organism(s)” means any organism capable of converting atmospheric nitrogen (N₂) into ammonia (NH₃).

As used herein, the term “nutrient(s)” means any nutrient (e.g., vitamins, macrominerals, micronutrients, trace minerals, organic acids, etc.) which are needed for plant growth, plant health, and/or plant development.

As used herein, the term “phosphate solubilizing organism” means any organism capable of converting insoluble phosphate into a soluble phosphate form.

As used herein, the terms “plant(s)” and “plant part(s)” means all plants and plant populations such as desired and undesired wild plants or crop plants (including naturally occurring crop plants). Crop plants can be plants, which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material and vegetative and generative propagation material (e.g., cuttings, tubers, rhizomes, off-shoots and seeds, etc.).

As used herein the terms “signal molecule(s)” or “plant signal molecule(s)”, which may be used interchangeably with “plant growth-enhancing agent(s),” broadly refer to any agent that results in increased or enhanced plant growth compared to untreated plants or plant parts (e.g., seeds and plants harvested from untreated seeds). Non-limiting examples of signal molecules include LCOs, COs, chitinous compounds, flavonoids, jasmonic acid or derivatives thereof, linoleic acid or derivatives thereof, linolenic acid or derivatives thereof, karrikins, etc.

As used herein, the terms “spore”, “microbial spore”, etc., has its normal meaning which is well known and understood by those of skill in the art. As used herein, the terms “spore” and “microbial spore” mean a microorganism in its dormant, protected state.

### Compositions

The compositions disclosed comprise a carrier, one or more agriculturally beneficial microorganisms as described herein, and one or more germinants. In certain embodiments, the composition may be in the form of a liquid, a gel, a slurry, a solid, or a powder (wettable powder or dry powder). In a particular embodiment, the composition is a dry or substantially dry composition. As used herein, the term “substantially dry composition(s)” is understood to be a composition containing less than 50 wt. % of free water, preferably less than 20 wt. % of free water, more preferably less than 10 wt. % of free water, even more preferably less than 5 wt. % of free water, still even more preferably less than 2.5 wt. % of free water, most preferably less than 1 wt. % of free water.

Dry compositions, as described herein, may be suitable for mixing with one or more liquids for formulation of a liquid product for foliar application to a plant or plant part, a seed treatment, an in furrow treatment, or a combination thereof. In yet another embodiment, the dry composition comprises microorganisms that remain in a spore form in the presence of a germinant until the dry composition is formulated (e.g., the composition is mixed and/or combined) with one or more solvents. Solvents may be aqueous or organic. Representative examples of solvents that may be suitable for use in certain embodiments include water or an organic solvent such as isopropyl alcohol or a glycol ether.

Carriers:

The carriers described herein will allow the microorganism(s) to remain efficacious (e.g., capable of enhancing plant growth, capable of expressing fungicidal activity, etc) and viable once formulated. Non-limiting examples of carriers described herein include liquids, slurries, or solids (including wettable powders or dry powders). In an embodiment, the carrier is a soil compatible carrier as described herein.

In one embodiment, the carrier is a liquid carrier. Non-limiting examples of liquids useful as carriers for the compositions disclosed herein include water, an aqueous solution, or a non-aqueous solution. In one embodiment, the carrier is water. In another embodiment the carrier is an aqueous solution, such as sugar water. In another embodiment, the carrier is a non-aqueous solution. If a liquid carrier is used, the liquid (e.g., water) carrier may further comprise growth media to culture the microorganisms described herein. Non-limiting examples of suitable growth media for the microorganisms described herein include arabinose-gluconate (AG), yeast extract mannitol (YEM), G16 media, or any media known to those skilled in the art to be compatible with, and/or provide growth nutrients to the strains.

In another embodiment, the carrier is a slurry. In an embodiment, the slurry may comprise a sticking agent, a liquid, or a combination thereof. It is envisioned that the sticking agent can be any agent capable of sticking the inoculum (e.g., one or more of the deposited strains) to a substrate of interest (e.g., a seed). Non-limiting examples of sticking agents include alginate, mineral oil, syrup, gum arabic, honey, methyl cellulose, milk, wallpaper paste, and combinations thereof. Non-limiting examples of liquids appropriate for a slurry include water or sugar water.

In another embodiment, the carrier is a solid. In a particular embodiment the solid is a powder. In one embodiment the powder is a wettable powder. In another embodiment, the powder is a dry powder. In another embodiment, the solid is a granule. Non-limiting examples of solids useful as carriers for the compositions disclosed herein include peat, wheat, wheat chaff, ground wheat straw, bran, vermiculite, cellulose, starch, soil (pasteurized or unpasteurized), gypsum, talc, clays (e.g., kaolin, bentonite, montmorillonite), and silica gels.

### Microorganisms:

The compositions disclosed herein comprise one or more microorganisms. In an embodiment, the one or more microorganisms are one or more bacteria. In another embodiment, the one or more microorganisms are one or more bacteria capable of having one or more beneficial properties to a plant and/or plant part (e.g., capable of promoting plant growth, capable of having fungicidal activity, etc.).

In a more particular embodiment, the one or more bacteria are spore forming bacterial strains. Methods for producing stabilized microorganisms, and bacteria specifically, are known in the art. See Donnellan, J. E., Nags, E. H., and Levinson, H. S. (1964). “Chemically defined, synthetic media for sporulation and for germination and growth of Bacillus subtilis.” Journal of Bacteriology 87(2):332-336; and Chen, Z., Li, Q., Liu, H. Yu, N., Xie, T., Yang, M., Shen, P., Chen, X. (2010). “Greater enhancement of Bacillus subtilis spore yields in submerged cultures by optimization of medium composition through statistical experimental designs.” Appl. Microbiol. Biotechnol. 85:1353-1360.

Non-limiting examples of spore forming bacterial strains include strains from the genera Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribaciflus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and/or Vulcanobacillus.

In a particular embodiment, the one or more spore forming bacteria is a bacteria selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibaciflus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof. In another embodiment, the one or more bacterial strains is a strain of Bacillus spp., e.g., Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, and combinations thereof.

In another embodiment, the one or more bacterial strains is a strain of Brevibacillus spp., e.g., Brevibacillus brevis; Brevibacillus formosus; Brevibacillus laterosporus; or Brevibacillus parabrevis, and combinations thereof.

In another embodiment, the one or more bacterial strains is a strain of Paenibacillus spp., e.g., Paenibacillus alvei; Paenibacillus amylolyticus; Paenibacillus azotofixans; Paenibacillus cookii; Paenibacillus macerans; Paenibacillus polymyxa; or Paenibacillus validus, and combinations thereof.

In a more particular embodiment, the one or more bacterial strains are a strain of Bacillus selected from the group consisting of Bacillus pumilus isolate AQ717, NRRL B-21662 (from Fa. AgraQuest Inc., USA), Bacillus pumilus isolate NRRL B-30087 (from Fa. AgraQuest Inc., USA), Bacillus sp., isolate AQ175, ATCC 55608 (from Fa. AgraQuest Inc., USA), Bacillus sp., isolate AQ177, ATCC 55609 (from Fa. AgraQuest Inc., USA), Bacillus subtilis isolate AQ713, NRRL B-21661 (in RHAPSODY®, SERENADE® MAX and SERENADE® ASO) (from Fa. AgraQuest Inc., USA), Bacillus subtilis isolate AQ743, NRRL B-21665 (from Fa. AgraQuest Inc., USA), Bacillus amyloliquefaciens FZB24 (e.g., deposited as isolates NRRL B-50304 and NRRL B-50349 TAEGRO® from Novozymes Biologicals, Inc., USA), Bacillus amyloliquefaciens TJ1000 (i.e., also known as 1BE, isolate ATCC BAA-390), Bacillus thuringiensis isolate AQ52, NRRL B-21619 (from Fa. AgraQuest Inc., USA), Bacillus subtilis var. amyloliquefaciens TrigoCor (also known as “TrigoCor 1448”; e.g., isolate Embrapa Trigo Accession No. 144/88.4Lev, Cornell Accession No. Pma007BR-97, and ATCC Accession No. 202152, from Cornell University, USA) and combinations thereof.

In a particular embodiment, the one or more bacterial strains will be present in a quantity between 1×10² and 1×10¹² CFU/g of the composition, particularly 1×10⁴ and 1×10¹¹ CFU/g of the composition, and more particularly 1×10⁵ and 5×10¹⁰ CFU/g of the composition. In a more particular embodiment the one or more bacterial strains will be present in a quantity between 1×10⁸ and 1×10¹⁰ CFU/g of the composition.

The fermentation of the one or more bacterial strains may be conducted using conventional fermentation processes, such as, aerobic liquid-culture techniques, shake flask cultivation, and small-scale or large-scale fermentation (e.g., continuous, batch, fed-batch, solid state fermentation, etc.) in laboratory or industrial fermentors, and such processes are well known in the art. Notwithstanding the production process used to produce the one or more bacterial strains, the one or more bacterial strains may be used directly from the culture medium or subject to purification and/or further processing steps (e.g., a drying process).

Following fermentation, the one or more bacterial strains may be recovered using conventional techniques (e.g., by filtration, centrifugation, etc.). The one or more bacterial strains may alternatively be dried (e.g., air-drying, freeze drying, or spray drying to a low moisture level, and storing at a suitable temperature, e.g., room temperature).

### Germinants:

The compositions described herein comprise one or more germinants. The one or more germinants described herein may be in either a liquid or solid form (including wettable powders or dry powders). In one embodiment, the germinant is in a liquid form. In another embodiment, the germinant is in a solid form. In a particular embodiment the germinant is a solid in the form of a powder. In another embodiment the powder is a wettable powder. In still another embodiment, the powder is a dry powder.

Non-limiting examples of germinants that may be suitable for the compositions described herein include lactate; lactose (as found in dairy products), bicarbonate or carbonate compounds such as sodium bicarbonate; carbon dioxide (e.g., carbonic acid: CO₂ dissolved in water, as is common in “sodas” or “soft drinks” such as cola or some fruit flavored beverages); compounds that adsorb lipid (e.g., starch, such as found in wheat, rice or other grains and potatoes and some other vegetables); charcoal or similar materials of high surface area that may adsorb or absorb fatty acid and lipid materials that may inhibit spore germination; monosaccharides such as fructose, glucose, mannose, or galactose; alanine, asparagine, cysteine, glutamine, norvatine, serine, threonine, valine, glycine, or other amino acid, and derivatives thereof such as N-(L-a-aspartyl)-L-phenylalanine (commonly sold under the trade name of “Aspartame”); inosine; bile salts such as taurocholate; and combinations of such spore germinants. For example, useful spore germinants can include alanine alone or in combination with lactate; a combination of L-asparagine, glucose, fructose, and potassium ion (AGFK); amino acids such as aspargine, cysteine, or serine alone or in combination with lactate; and caramels created by autoclaving monosaccharides or such caramels in combination with amino acids. In one embodiment, the composition comprises one or more germinants. In a particular embodiment, the composition comprises L-asparagine, glucose, fructose, and potassium ion (AGFK).

In a particular embodiment, the one or more germinants will be present in a concentration of 0.001 mM to 10.0 M of the composition, particularly 0.01 mM to 5.0 M of the composition, and more particularly 0.1 mM to 1.0 M of the composition. In a more particular embodiment the one or more germinants will be present in a concentration between 1.0 mM to 0.1 M of the composition.

### Agriculturally Beneficial Ingredients:

The compositions disclosed herein may comprise one or more agriculturally beneficial ingredients. Alternatively, as persons skilled in the art would appreciate, any one or more of these agents may be used in the methods described herein via separate composition or formulation. Non-limiting examples of agriculturally beneficial ingredients include one or more biologically active ingredients, nutrients, biostimulants, preservatives, polymers, wetting agents, surfactants, herbicides, fungicides, insecticides, or combinations thereof.

Biologically Active Ingredient(s):

The compositions described herein may optionally include one or more biologically active ingredients as described herein, other than the one or more flavonoids described herein. Non-limiting examples of biologically active ingredients include plant signal molecules (e.g., lipo-chitooligosaccharides (LCO), chitooligosaccharides (CO), chitinous compounds, jasmonic acid or derivatives thereof, linoleic acid or derivatives thereof, linolenic acid or derivatives thereof, karrikins, etc.) and beneficial microorganisms (e.g., Rhizobium spp., Bradyrhizobium spp., Sinorhizobium spp., Azorhizobium spp., Glomus spp., Gigaspora spp., Hymenoscyphous spp., Oidiodendron spp., Laccaria spp., Pisolithus spp., Rhizopogon spp., Scleroderma spp., Rhizoctonia spp., Acinetobacter spp., Arthrobacter spp., Arthrobotrys spp., Aspergillus spp., Azospirillum spp, Bacillus spp, Burkholderia spp., Candida spp., Chryseomonas spp., Enterobacter spp., Eupenicillium spp., Exiguobacterium spp., Klebsiella spp., Kluyvera spp., Microbacterium spp., Mucor spp., Paecilomyces spp., Paenibacillus spp., Penicillium spp., Pseudomonas spp., Serratia spp., Stenotrophomonas spp., Streptomyces spp., Streptosporangium spp., Swaminathania spp., Thiobacillus spp., Torulospora spp., Vibrio spp., Xanthobacter spp., Xanthomonas spp., etc.).

Plant Signal Molecule(s):

In an embodiment, the compositions described herein may include one or more plant signal molecules. In one embodiment, the one or more plant signal molecules are one or more LCOs. In another embodiment, the one or more plant signal molecules are one or more COs. In still another embodiment, the one or more plant signal molecules are one or more chitinous compounds. In yet another embodiment, the one or more plant signal molecules are one or more non-flavonoid nod gene inducers (e.g., jasmonic acid, linoleic acid, linolenic acid, and derivatives thereof). In still yet another embodiment, the one or more plant signal molecules are one or more karrikins or derivatives thereof. In still another embodiment, the one or more plant signal molecules are one or more LCOs, one or more COs, one or more chitinous compounds, one or more non-flavonoid nod gene inducers and derivatives thereof, one or more karrikins and derivatives thereof, or any signal molecule combination thereof.

LCOs:

Lipo-chitooligosaccharide compounds (LCOs), also known as symbiotic Nod signals or Nod factors, consist of an oligosaccharide backbone of β-1,4-linked N-acetyl-D-glucosamine (“GlcNAc”) residues with an N-linked fatty acyl chain condensed at the non-reducing end. LCO's differ in the number of GlcNAc residues in the backbone, in the length and degree of saturation of the fatty acyl chain, and in the substitutions of reducing and non-reducing sugar residues. See, e.g., Denarie, et al., Ann. Rev. Biochem. 65:503-35 (1996), Hamel, et al., Planta 232:787-806 (2010); Prome, et al., Pure & Appl. Chem. 70(1):55-60 (1998). An example of an LCO is presented below as formula I:

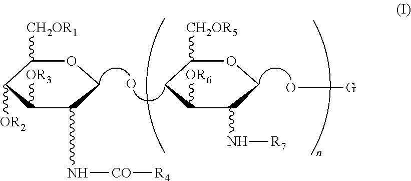

in which:

G is a hexosamine which can be substituted, for example, by an acetyl group on the nitrogen, a sulfate group, an acetyl group and/or an ether group on an oxygen,

R₁, R₂, R₃, R₅, R₆ and R₇, which may be identical or different, represent H, CH₃CO—, CₓH_{y} CO— where x is an integer between 0 and 17, and y is an integer between 1 and 35, or any other acyl group such as for example a carbamoyl,

R₄ may represent a fully saturated aliphatic chain containing at least 12 carbon atoms or may represent a mono-, di- or triunsaturated aliphatic chain containing at least 12 carbon atoms, and n is an integer between 1 and 4.

LCOs may be obtained (isolated and/or purified) from bacteria such as Rhizobia, e.g., Rhizobium sp., Bradyrhizobium sp., Sinorhizobium sp. and Azorhizobium sp. LCO structures are characteristic for each such bacterial species, and each strain may produce multiple LCO's with different structures. For example, specific LCOs from S. meliloti have also been described in U.S. Pat. No. 5,549,718 as having the formula II:

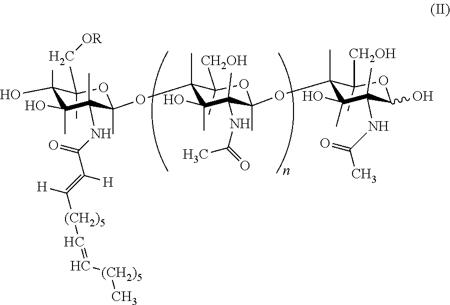

in which R represents H or CH₃CO— and n is equal to 2 or 3.

Even more specific LCOs include NodRM, NodRM-1, NodRM-3. When acetylated (the R═CH₃CO—), they become AcNodRM-1, and AcNodRM-3, respectively (U.S. Pat. No. 5,545,718).

LCOs from Bradyrhizobium japonicum are described in U.S. Pat. Nos. 5,175,149 and 5,321,011. Broadly, they are pentasaccharide phytohormones comprising methylfucose. A number of these B. japonicum-derived LCOs are described: BjNod-V (C_{18:1}); BjNod-V (A_{C}, C_{18:1}), BjNod-V (C_{16:1}); and BjNod-V (A_{C}, C_{16:0}), with “V” indicating the presence of five N-acetylglucosamines; “Ac” an acetylation; the number following the “C” indicating the number of carbons in the fatty acid side chain; and the number following the “:” the number of double bonds.

LCO's used in embodiments of the invention may be obtained (i.e., isolated and/or purified) from bacterial strains that produce LCO's, such as strains of Azorhizobium, Bradyrhizobium (including B. japonicum), Mesorhizobium, Rhizobium (including R. leguminosarum), Sinorhizobium (including S. meliloti), and bacterial strains genetically engineered to produce LCO's. In some embodiments, there is a combination of two or more LCO's obtained from these rhizobial and bradyrhizobial microorganisms.

LCO's are the primary determinants of host specificity in legume symbiosis (Diaz, et al., Mol. Plant-Microbe Interactions 13:268-276 (2000)). Thus, within the legume family, specific genera and species of rhizobia develop a symbiotic nitrogen-fixing relationship with a specific legume host. These plant-host/bacteria combinations are described in Hungria, et al., Soil Biol. Biochem. 29:819-830 (1997), Examples of these bacteria/legume symbiotic partnerships include S. melilotilalfalfa and sweet clover; R. leguminosarum biovar viciae/peas and lentils; R. leguminosarum biovar phaseoli/beans; Bradyrhizobium japonicum/soybeans; and R. leguminosarum biovar trifolii/red clover. Hungria also lists the effective flavonoid Nod gene inducers of the rhizobial species, and the specific LCO structures that are produced by the different rhizobial species. However, LCO specificity is only required to establish nodulation in legumes. Use of a given LCO is not limited to treatment of seed of its symbiotic legume partner in order to achieve increased plant yield measured in terms of bushels/acre, increased root number, increased root length, increased root mass, increased root volume and increased leaf area, compared to plants harvested from untreated seed.

Thus, by way of further examples, LCO's as well as naturally and non-naturally occurring derivatives thereof that may be useful in some embodiments are represented by the following formula:

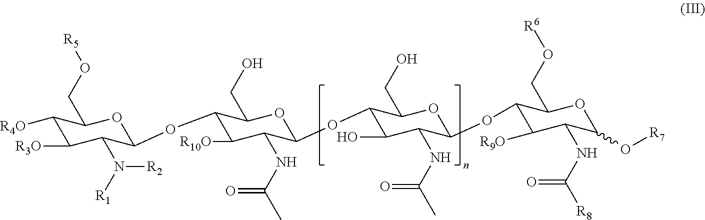

wherein R₁ represents C14:0, 3OH—C14:0, iso-C15:0, C16:0, 3-OH—C16:0, iso-C15:0, C16:1, C16:2, C16:3, iso-C17:0, iso-C17:1, C18:0, 3OH—C18:0, C18:0/3-OH, C18:1, OH—C18:1, C18:2, C18:3, C18:4, C19:1 carbamoyl, C20:0, C20:1, 3-OH—C20:1, C20:1/3-OH, C20:2, C20:3, C22:1, and C18-26(ω-1)-OH (which according to D'Haeze, et al., Glycobiology 12:79R-105R (2002), includes C18, C20, C22, C24 and C26 hydroxylated species and C16:1Δ9, C16:2 (Δ2,9) and C16:3 (Δ2,4,9)); R₂ represents hydrogen or methyl; R₃ represents hydrogen, acetyl or carbamoyl; R₄ represents hydrogen, acetyl or carbamoyl; R₅ represents hydrogen, acetyl or carbamoyl; R₆ represents hydrogen, arabinosyl, fucosyl, acetyl, SO₃H, sulfate ester, 3-0-S-2-0-MeFuc, 2-0-MeFuc, and 4-0-AcFuc; R₇ represents hydrogen, mannosyl or glycerol; R₈ represents hydrogen, methyl, or —CH₂OH; R₉ represents hydrogen, arabinosyl, or fucosyl; R₁₀ represents hydrogen, acetyl or fucosyl; and n represents 0, 1, 2 or 3. The structures of the naturally occurring Rhizobial LCO's embraced by this structure are described in D'Haeze, et al., supra.

Also encompassed in some embodiments is use of LCO's obtained (i.e., isolated and/or purified) from a mycorrhizal fungi, such as fungi of the group Glomerocycota, e.g., Glomus intraradicus. The structures of representative LCOs obtained from these fungi are described in WO 2010/049751 and WO 2010/049751 (the LCOs described therein also referred to as “Myc factors”). Representative mycorrhizal fungi-derived LCO's and non-naturally occurring derivatives thereof are represented by the following structure:

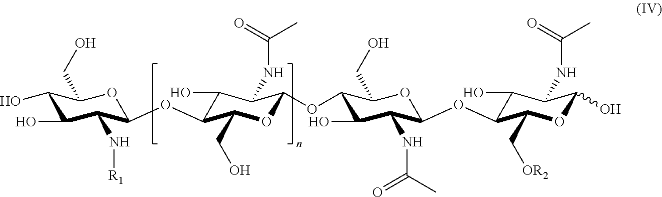

wherein n=1 or 2; R₁ represents C16, C16:0, C16:1, C16:2, C18:0, C18:1Δ9Z or C18:1Δ11Z; and R₂ represents hydrogen or SO₃H. In some embodiments, the LCO's are produced by the mycorrhizal fungi. In some embodiments, these LCO's are used in the methods described herein.

Further encompassed in some embodiments described herein is use of synthetic LCO compounds, such as those described in WO 2005/063784, chemically synthesized LCO compounds, such as those described in WO 2007/117500, and recombinant LCO's produced through genetic engineering. The basic, naturally occurring LCO structure may contain modifications or substitutions found in naturally occurring LCO's, such as those described in Spaink, Crit. Rev. Plant Sci. 54:257-288 (2000) and D'Haeze, supra. Precursor oligosaccharide molecules (COs, which as described below, are also useful as plant signal molecules) for the construction of LCOs may also be synthesized by genetically engineered organisms, e.g., as described in Samain, et al., Carbohydrate Res. 302:35-42 (1997); Cottaz, et al., Meth. Eng. 7(4):311-7 (2005) and Samain, et al., J. Biotechnol. 72:33-47 (1999)(e.g., FIG. 1 therein which shows structures of CO's that can be made recombinantly in E. coli harboring different combinations of genes nodBCHL).

LCO's may be utilized in various forms of purity and may be used alone or in the form of a culture of LCO-producing bacteria or fungi. For example, OPTIMIZE® (commercially available from Novozymes BioAg Inc.) contains a culture of B. japonicum that produces an LCO (LCO-V(C18:1, MeFuc), MOR116). Methods to provide substantially pure LCO's include simply removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC, and the purified LCO molecules can be freeze-dried for long-term storage. Chitooligosaccharides (COs), may be used as starting materials for the production of synthetic LCOs. For the purposes of some embodiments, recombinant LCO's suitable for use are least 60% pure, e.g., at least 60% pure, at least 65% pure, at least 70% pure, at least 75% pure, at least 80% pure, at least 85% pure, at least 90% pure, at least 91% pure, at least 92% pure, at least 93% pure, at least 94% pure, at least 95% pure, at least 96% pure, at least 97% pure, at least 98% pure, at least 99% pure, up to 100% pure.

COs:

Chitooligosaccharides (COs) are known as β-1-4 linked N actyl glucosamine structures identified as chitin oligomers, also as N-acetylchitooligosaccharides. CO's have unique and different side chain decorations which make them different from chitin molecules [(C₈H₁₃NO₅)n, CAS No. 1398-61-4], and chitosan molecules [(C₅H₁₁NO₄)n, CAS No. 9012-76-4]. Representative literature describing the structure and production of COs is as follows: Van der Holst, et al., Current Opinion in Structural Biology, 11:608-616 (2001); Robina, et al., Tetrahedron 58:521-530 (2002); Hanel, et al., Planta 232:787-806 (2010); Rouge, et al. Chapter 27, “The Molecular Immunology of Complex Carbohydrates” in Advances in Experimental Medicine and Biology, Springer Science; Wan, et al., Plant Cell 21:1053-69 (2009); PCT/F100/00803 (Sep. 21, 2000); and Demont-Caulet, et al., Plant Physiol. 120(1):83-92 (1999). The COs may be synthetic or recombinant. Methods for preparation of recombinant COs are known in the art. See, e.g., Samain, et al. (supra.); Cottaz, et al., Meth. Eng. 7(4):311-7 (2005) and Samain, et al., J. Biotechnol. 72:33-47 (1999). COs are intended to include isomers, salts, and solvates thereof.

Chitinous Compounds:

Chitins and chitosans, which are major components of the cell walls of fungi and the exoskeletons of insects and crustaceans, are also composed of GlcNAc residues. Chitinous compounds include chitin, (IUPAC: N-[5-[[3-acetylamino-4,5-dihydroxy-6-(hydroxymethyl)oxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan, (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol), and isomers, salts, and solvates thereof.

Certain chitins and chitosan compounds may be obtained commercially, e.g., from Sigma-Aldrich, or prepared from insects, crustacean shells, or fungal cell walls. Methods for the preparation of chitin and chitosan are known in the art, and have been described, for example, in U.S. Pat. No. 4,536,207 (preparation from crustacean shells), Pochanavanich, et al., Lett. Appl. Microbiol. 35:17-21 (2002) (preparation from fungal cell walls), and U.S. Pat. No. 5,965,545 (preparation from crab shells and hydrolysis of commercial chitosan). Deacetylated chitins and chitosans may be obtained that range from less than 35% to greater than 90% deacetylation, and cover a broad spectrum of molecular weights, e.g., low molecular weight chitosan oligomers of less than 15 kD and chitin oligomers of 0.5 to 2 kD; “practical grade” chitosan with a molecular weight of about 15 kD; and high molecular weight chitosan of up to 70 kD. Certain chitin and chitosan compositions formulated for seed treatment are also commercially available. Commercial products include, for example, ELEXA® (Plant Defense Boosters, Inc.) and BEYOND™ (Agrihouse, Inc.).

Flavonoids (Nod-Gene Inducers):

Flavonoid compounds are commercially available, e.g., from Novozymes BioAg, Saskatoon, Canada; Natland International Corp., Research Triangle Park, N.C.; MP Biomedicals, Irvine, Calif.; LC Laboratories, Woburn Mass. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, as described in Ralston, et al., Plant Physiology 137:1375-88 (2005). Flavonoid compounds are intended to include all flavonoid compounds as well as isomers, salts, and solvates thereof.

The one or more flavonoids may be a natural flavonoid (i.e., not synthetically produced), a synthetic flavonoid (e.g., a chemically synthesized flavonoid) or a combination thereof. In a particular embodiment, the compositions described herein may comprise a flavanol, a flavone, an anthocyanidin, an isoflavonoid, a neoflavonoid and combinations thereof, including all isomer, solvate, hydrate, polymorphic, crystalline form, non-crystalline form, and salt variations thereof.

In an embodiment, the compositions described herein may comprise one or more flavanols. In still another embodiment, the compositions described herein may comprise one or more flavanols selected from the group consisting of flavan-3-ols (e.g., catechin (C), gallocatechin (GC), catechin 3-gallate (Cg), gallcatechin 3-gallate (GCg), epicatechins (EC), epigallocatechin (EGC) epicatechin 3-gallate (ECg), epigallcatechin 3-gallate (EGCg), etc.), flavan-4-ols, flavan-3,4-diols (e.g., leucoanthocyanidin), proanthocyanidins (e.g., includes dimers, trimers, oligomers, or polymers of flavanols), and combinations thereof. In still yet another embodiment, the compositions may comprise one or more flavanols selected from the group consisting of catechin (C), gallocatechin (GC), catechin 3-gallate (Cg), gallcatechin 3-gallate (GCg), epicatechins (EC), epigallocatechin (EGC) epicatechin 3-gallate (ECg), epigallcatechin 3-gallate (EGCg), flavan-4-ol, leucoanthocyanidin, and dimers, trimers, olilgomers or polymers thereof.

In another embodiment, the compositions described herein may comprise one or more flavones. In still another embodiment, the compositions described herein may comprise one or more flavones selected from the group consisting of flavones (e.g., luteolin, apigenin, tangeritin, etc.), flavonols (e.g., quercetin, quercitrin, rutin, kaempferol, kaempferitrin, astragalin, sophoraflavonoloside, myricetin, fisetin, isorhamnetin, pachypodol, rhamnazin, etc.), flavanones (e.g. hesperetin, hesperidin, naringenin, eriodictyol, homoeriodictyol, etc.), and flavanonols (e.g., dihydroquercetin, dihydrokaempferol, etc.). In still yet another embodiment, the compositions may comprise one or more flavones selected from the group consisting of luteolin, apigenin, tangeritin, quercetin, quercitrin, rutin, kaempferol, kaempferitrin, astragalin, sophoraflavonoloside, myricetin, fisetin, isorhamnetin, pachypodol, rhamnazin, hesperetin, hesperidin, naringenin, eriodictyol, homoeriodictyol, dihydroquercetin, dihydrokaempferol, and combinations thereof.

In still another embodiment, the compositions described herein may comprise one or more anthocyanidins. In yet another embodiment, the compositions described herein may comprise one or more anthocyanidins selected from the group consisting of cyanidins, delphinidins, malvidins, pelargonidins, peonidins, petunidins, and combinations thereof.

In another embodiment, the compositions described herein may comprise one or more isoflavonoids. In still yet another embodiment, the compositions described herein may comprise one or more isoflavonoids selected from the group consisting of phytoestrogens, isoflavones (e.g., genistein, daidzein, glycitein, etc.), and isoflavanes (e.g., equol, lonchocarpane, laxiflorane, etc.), and combinations thereof. In yet another embodiment the compositions may comprise one or more isoflavonoids selected from the group consisting of genistein, daidzein, glycitein, equol, lonchocarpane, laxiflorane, and combinations thereof.

In another embodiment, the compositions described herein may comprise one or more neoflavonoids. In yet another embodiment, the compositions described herein may comprise one or more neoflavonoids selected from the group consisting of neoflavones (e.g., calophyllolide), neoflavenes (e.g., dalbergichromene), coutareagenins, dalbergins, nivetins, and combinations thereof. In still yet another embodiment, the compositions described herein may comprise one or more neoflavonoids selected from the group consisting of calophyllolide, dalbergichromene, coutareagenin, dalbergin, nivetin, and combinations thereof.

Non-Flavonoid Nod-Gene Inducer(s):

Jasmonic acid (JA, [1R-[1α,2β(Z)]]-3-oxo-2-(pentenyl)cyclopentaneacetic acid) and its derivatives, linoleic acid ((Z,Z)-9,12-Octadecadienoic acid) and its derivatives, and linolenic acid ((Z,Z,Z)-9,12,15-octadecatrienoic acid) and its derivatives, may also be used in the compositions described herein. Non-flavonoid nod-gene inducers are intended to include not only the non-flavonoid nod-gene inducers described herein, but isomers, salts, and solvates thereof.

Jasmonic acid and its methyl ester, methyl jasmonate (MeJA), collectively known as jasmonates, are octadecanoid-based compounds that occur naturally in plants. Jasmonic acid is produced by the roots of wheat seedlings, and by fungal microorganisms such as Botryodiplodia theobromae and Gibberella fujikuroi, yeast (Saccharomyces cerevisiae), and pathogenic and non-pathogenic strains of Escherichia coli. Linoleic acid and linolenic acid are produced in the course of the biosynthesis of jasmonic acid. Jasmonates, linoleic acid and linoleic acid (and their derivatives) are reported to be inducers of nod gene expression or LCO production by rhizobacteria. See, e.g., Mabood, Fazli, Jasmonates induce the expression of nod genes in Bradyrhizobium japonicum, May 17, 2001; and Mabood, Fazli, “Linoleic and linolenic acid induce the expression of nod genes in Bradyrhizobium japonicum,” USDA 3, May 17, 2001.

Useful derivatives of linoleic acid, linolenic acid, and jasmonic acid that may be useful in compositions described herein include esters, amides, glycosides and salts. Representative esters are compounds in which the carboxyl group of linoleic acid, linolenic acid, or jasmonic acid has been replaced with a —COR group, where R is an —OR¹ group, in which R¹ is: an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are independently: hydrogen; an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic addition, wherein the carboxylic acid is reacted with an alcohol in the presence of a catalytic amount of a mineral acid. Amides may also be prepared by known methods, such as by reacting the carboxylic acid with the appropriate amine in the presence of a coupling agent such as dicyclohexyl carbodiimide (DCC), under neutral conditions. Suitable salts of linoleic acid, linolenic acid, and jasmonic acid include e.g., base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing together a solution of linoleic acid, linolenic acid, or jasmonic acid with a solution of the base. The salt may be precipitated from solution and be collected by filtration or may be recovered by other means such as by evaporation of the solvent.

Karrikin(s):

Karrikins are vinylogous 4H-pyrones e.g., 2H-furo[2,3-c]pyran-2-ones including derivatives and analogues thereof. It is intended that the karrikins include isomers, salts, and solvates thereof. Examples of these compounds are represented by the following structure:

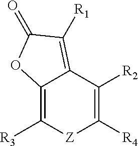

wherein; Z is O, S or NR₅; R₁, R₂, R₃, and R₄ are each independently H, alkyl, alkenyl, alkynyl, phenyl, benzyl, hydroxy, hydroxyalkyl, alkoxy, phenyloxy, benzyloxy, CN, COR₆, COOR═, halogen, NR₆R₇, or NO₂; and R₅, R₆, and R₇ are each independently H, alkyl or alkenyl, or a biologically acceptable salt thereof. Examples of biologically acceptable salts of these compounds may include acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate and p-toluenesulphonic acid. Additional biologically acceptable metal salts may include alkali metal salts, with bases, examples of which include the sodium and potassium salts. Examples of compounds embraced by the structure and which may be suitable for use in some embodiments described herein include the following: 3-methyl-2H-furo[2,3-c]pyran-2-one (where R₁═CH₃, R₂, R₃, R₄═H), 2H-furo[2,3-c]pyran-2-one (where R₁, R₂, R₃, R4═H), 7-methyl-2H-furo[2,3-c]pyran-2-one (where R₁, R₂, R₄═H, R₃═CH₃), 5-methyl-2H-furo[2,3-c]pyran-2-one (where R₁, R₂, R₃═H, R₄═CH₃), 3,7-dimethyl-2H-furo[2,3-c]pyran-2-one (where R₁, R₃═CH₃, R₂, R₄═H), 3,5-dimethyl-2H-furo[2,3-c]pyran-2-one (where R₁, R₄═CH₃, R₂, R₃═H), 3,5,7-trimethyl-2H-furo[2,3-c]pyran-2-one (where R₁, R₃, R₄═CH₃, R₂═H), 5-methoxymethyl-3-methyl-2H-furo[2,3-c]pyran-2-one (where R₁═CH₃, R₂, R₃═H, R₄═CH₂OCH₃), 4-bromo-3,7-dimethyl-2H-furo[2,3-c]pyran-2-one (where R₁, R₃═CH₃, R₂═Br, R₄═H), 3-methylfuro[2,3-c]pyridin-2(3H)-one (where Z═NH, R₁═CH₃, R₂, R₃, R₄═H), 3,6-dimethylfuro[2,3-c]pyridin-2(6H)-one (where Z═N—CH₃, R₁═CH₃, R₂, R₃, R₄═H). See, U.S. Pat. No. 7,576,213. These molecules are also known as karrikins. See, Halford, “Smoke Signals,” in Chem. Eng. News (Apr. 12, 2010), at pages 37-38 (reporting that karrikins or butenolides which are contained in smoke act as growth stimulants and spur seed germination after a forest fire, and can invigorate seeds such as corn, tomatoes, lettuce and onions that had been stored). These molecules are the subject of U.S. Pat. No. 7,576,213.

Beneficial Microorganism(s):

In an embodiment, the compositions described herein may comprise one or more additional beneficial microorganisms other than those previously described. The one or more beneficial microorganisms may be in a spore form, a vegetative form, or a combination thereof.

A) Diazotrophs:

In particular embodiments, the one or more beneficial microorganisms are diazotrophs (i.e., bacteria which are symbiotic nitrogen-fixing bacteria).

In embodiments, the diazotroph is a bacterium of the genus Azorhizobium, Azospirillum, Bradyrhizobium, Mesorhizobium, Rhizobium, Sinorhizobium, and combinations thereof.

Non-limiting examples of particular species that may be useful as a bacterial diazotroph in the compositions described herein include Azorhizobium caulinodans, Azorhizobium doebereinerae, Azospirillum amazonense, Azospirillum brasilense, Azospirillum brasilense isolate INTA Az-39 (available from Novozymes), Azospirillum canadense, Azospirillum doebereinerae, Azospirillum formosense, Azospirillum halopraeferans, Azospirillum irakense, Azospirillum largimobile, Azospirillum lipoferum, Azospirillum melinis, Azospirillum oryzae, Azospirillum picis, Azospirillum rugosum, Azospirillum thiophilum, Azospirillum zeae, Bradyrhizobium bête, Bradyrhizobium canariense, Bradyrhizobium elkanii, Bradyrhizobium elkanii isolate SEMIA 587 (available from Novozymes), Bradyrhizobium elkanii isolate SEMIA 5019 (available from Novozymes), Bradyrhizobium iriomotense, Bradyrhizobium japonicum, Bradyrhizobium japonicum isolate SEMIA 5079 (available from Novozymes), Bradyrhizobium japonicum isolate SEMIA 5080 (available from Novozymes), Bradyrhizobium japonicum isolate NRRL B-50608 (available from Novozymes), Bradyrhizobium japonicum isolate NRRL B-50609 (available from Novozymes), Bradyrhizobium japonicum isolate NRRL B-50610 (available from Novozymes), Bradyrhizobium japonicum isolate NRRL B-50611 (available from Novozymes), Bradyrhizobium japonicum isolate NRRL B-50612 (available from Novozymes), Bradyrhizobium japonicum isolate NRRL B-50592 (deposited also as NRRL B-59571) (available from Novozymes), Bradyrhizobium japonicum isolate NRRL B-50593 (deposited also as NRRL B-59572) (available from Novozymes), Bradyrhizobium japonicum isolate NRRL B-50586 (deposited also as NRRL B-59565) (available from Novozymes), Bradyrhizobium japonicum isolate NRRL B-50588 (deposited also as NRRL B-59567) (available from Novozymes), Bradyrhizobium japonicum isolate NRRL B-50587 (deposited also as NRRL B-59566) (available from Novozymes), Bradyrhizobium japonicum isolate NRRL B-50589 (deposited also as NRRL B-59568) (available from Novozymes), Bradyrhizobium japonicum isolate NRRL B-50591 (deposited also as NRRL B-59570) (available from Novozymes), Bradyrhizobium japonicum NRRL B-50590 (deposited also as NRRL B-59569) (available from Novozymes), Bradyrhizobium japonicum isolate NRRL B-50594 (deposited also as NRRL B-50493) (available from Novozymes), Bradyrhizobium japonicum isolate NRRL B-50726 (available from Novozymes), Bradyrhizobium japonicum isolate NRRL B-50727 (available from Novozymes), Bradyrhizobium japonicum isolate NRRL B-50728 (available from Novozymes), Bradyrhizobium japonicum isolate NRRL B-50729 (available from Novozymes), Bradyrhizobium japonicum isolate NRRL B-50730 (available from Novozymes), Bradyrhizobium japonicum isolate USDA 532C, Bradyrhizobium japonicum isolate USDA 110, Bradyrhizobium japonicum isolate USDA 123, Bradyrhizobium japonicum isolate USDA 127, Bradyrhizobium japonicum isolate USDA 129, Bradyrhizobium jicamae, Bradyrhizobium liaoningense, Bradyrhizobium pachyrhizi, Bradyrhizobium yuanmingense, Mesorhizobium albiziae, Mesorhizobium amorphae, Mesorhizobium chacoense, Mesorhizobium ciceri, Mesorhizobium huakuii, Mesorhizobium loti, Mesorhizobium mediterraneum, Mesorhizobium pluifarium, Mesorhizobium septentrionale, Mesorhizobium temperatum, Mesorhizobium tianshanense, Rhizobium cellulosilyticum, Rhizobium daejeonense, Rhizobium etli, Rhizobium galegae, Rhizobium gallicum, Rhizobium giardinii, Rhizobium hainanense, Rhizobium huautlense, Rhizobium indigoferae, Rhizobium leguminosarum, Rhizobium leguminosarum isolate SO12A-2-(IDAC 080305-01), Rhizobium loessense, Rhizobium lupini, Rhizobium lusitanum, Rhizobium meliloti, Rhizobium mongolense, Rhizobium miluonense, Rhizobium sullae, Rhizobium tropici, Rhizobium undicola, Rhizobium yanglingense, Sinorhizobium abri, Sinorhizobium adhaerens, Sinorhizobium americanum, Sinorhizobium aboris, Sinorhizobium fredii, Sinorhizobium indiaense, Sinorhizobium kostiense, Sinorhizobium kummerowiae, Sinorhizobium medicae, Sinorhizobium meliloti, Sinorhizobium mexicanus, Sinorhizobium morelense, Sinorhizobium saheli, Sinorhizobium terangae, Sinorhizobium xinjiangense, and combinations thereof.

B) Phosphate Solubilizing Microorganisms:

In particular embodiments, the one or more beneficial microorganisms are phosphate solubilizing microorganisms.

In embodiments, the phosphate solubilizing microorganism is a fungus of the genus Penicillium, Talaromyces, and combinations thereof.

Non-limiting examples of particular species that may be useful as a phosphate solubilizing fungus in the compositions described herein include Penicillium albidum, Penicillium aurantiogriseum, Penicillium bilaiae (formerly known as Penicillium bilaii and Penicillium bilaji), Penicillium bilaiae isolate ATCC 20851, Penicillium bilaiae isolate ATCC 22348, Penicillium bilaiae isolate V08/021001 (also deposited as NRRL B-50612), Penicillium bilaiae isolate NRRL B-50776, Penicillium bilaiae isolate NRRL B-50777, Penicillium bilaiae isolate NRRL B-50778, Penicillium bilaiae isolate NRRL B-50779, Penicillium bilaiae isolate NRRL B-50780, Penicillium bilaiae isolate NRRL B-50781, Penicillium bilaiae isolate NRRL B-50782, Penicillium bilaiae isolate NRRL B-50783, Penicillium bilaiae isolate NRRL B-50784, Penicillium bilaiae isolate NRRL B-50785, Penicillium bilaiae isolate NRRL B-50786, Penicillium bilaiae isolate NRRL B-50787, Penicillium bilaiae isolate NRRL B-50788, Penicillium bilaiae isolate NRRL B-50169, Penicillium bilaiae isolate ATCC 18309, Penicillium brevicompactum, Penicillium brevicompactum isolate AgRF18, Penicillium canescens, Penicillium canescens isolate ATCC 10419, Penicillium chrysogenum, Penicillium citreonigrum, Penicillium citrinum, Penicillium digitatum, Penicillium expansum, Penicillium expansum isolate ATCC 24692, Penicillium expansum isolate YT02, Penicillium fellutanum, Penicillium fellutanum isolate ATCC 48694, Penicillium frequentas, Penicillium fuscum, Penicillium fussiporus, Penicillium gaestrivorus, Penicillium gaestrivorus isolate NRRL 50170, Penicillium glabrum, Penicillium glabrum isolate DAOM 239074, Penicillium glabrum isolate CBS 229.28, Penicillium glaucum, Penicillium griseofulvum, Penicillium implicatum, Penicillium janthinellum, Penicillium janthinellum isolate ATCC 10455, Penicillium lanosocoeruleum, Penicillium lanosocoeruleum isolate ATCC 48919, Penicillium lilacinum, Penicillium minioluteum, Penicillium montanense, Penicillium nigricans, Penicillium oxalicum, Penicillium pinetorum, Penicillium pinophilum, Penicillium purpurogenum, Penicillium radicum, Penicillium radicum isolate N93/47267, Penicillium radicum isolate FRR 4717, Penicillium radicum isolate ATCC 201836, Penicillium radicum isolate FRR 4719, Penicillium raistrickii, Penicillium raistrickii isolate ATCC 10490, Penicillium rugulosum, Penicillium simplicissimum, Penicillium solitum, Penicillium variabile, Penicillium velutinum, Penicillium viridicatum, Talaromyces aculeatus, Talaromyces aculeatus isolate ATCC 10409, and combinations thereof.

C) Mycorrhiza:

In particular embodiments, the one or more beneficial microorganisms are mycorrhiza. Suitable mycorrhizas include endomycorrhiza (also called vesicular arbuscular mycorrhizas, VAMs, arbuscular mycorrhizas, or AMs), ectomycorrhizas, ericoid mycorrhizas, and combinations thereof.

In embodiments, the mycorrhiza is a fungus of the genus Gigaspora, Glomus, Hymenoscyphous, Laccaria, Oidiodendron, Paraglomus, Pisolithus, Rhizoctonia, Rhizopogon, Scleroderma, and combinations thereof.

Non-limiting examples of particular mycorrhizal species that may be useful in the compositions described herein include Gigaspora margarita, Glomus aggregatum, Glomus brasilianum, Glomus clarum, Glomus deserticola, Glomus etunicatum, Glomus fasciculatum, Glomus intraradices, Glomus monosporum, Glomus mosseae, Hymenoscyphous ericae, Laccaria bicolor, Laccaria laccata, Oidiodendron sp., Paraglomus brazilianum, Pisolithus tinctorius, Rhizoctonia sp., Rhizopogon amylopogon, Rhizopogon fulvigleba, Rhizopogon luteolus, Rhizopogon villosuli, Scleroderma cepa, Scleroderma citrinum, Rhizoplex® (Gigaspora margarita, Glomus aggregatum, Glomus brasilianum, Glomus clarum, Glomus deserticola, Glomus etunicatum, Glomus intraradices, Glomus monosporum, Glomus mosseae, Laccaria bicolor, Laccaria laccata, Pisolithus tinctorius, Rhizopogon amylopogon, Rhizopogon fulvigleba, Rhizopogon luteolus, Rhizopogon villosuli, Scleroderma cepa and Scleroderma citrinum) (available from Novozymes), Rhizomyco® (Gigaspora margarita, Glomus aggregatum, Glomus clarum, Glomus deserticola, Glomus etunicatum, Glomus intraradices, Glomus monosporum, Glomus mosseae, Laccaria bicolor, Laccaria laccata, Paraglomus brazilianum, Pisolithus tinctorius, Rhizopogon amylopogon, Rhizopogon fulvigleba, Rhizopogon luteolus, Rhizopogon villosuli, Scleroderma cepa and Scleroderma citrinum) (available from Novozymes), Rhizomyx® (Gigaspora margarita, Glomus aggregatum, Glomus brasilianum, Glomus clarum, Glomus deserticola, Glomus etunicatum, Glomus intraradices, Glomus monosporum, and Glomus mosseae) (available from Novozymes), and combinations thereof.

In still another embodiment, the one or more beneficial microorganisms are microorganisms capable of exhibiting fungicidal activity, (e.g., biofungicides). Non-limiting examples of biofungicides are described in the “Fungicides” section below.

Herbicide(s):

In one embodiment, the compositions described herein may further comprise one or more herbicides.

Non-limiting examples of herbicides may acetyl CoA carboxylase (ACCase) inhibitors, acetolactate synthase (ALS) or acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvyl shikimate-3-phosphate (EPSP) synthase inhibitor, glutamine synthetase inhibitor, dihydropteroate synthetase inhibitor, mitosis inhibitors, 4-hydroxyphenyl-pyruvate-dioxygenase (4-HPPD) inhibitors, synthetic auxins, auxin herbicide salts, auxin transport inhibitors, and nucleic acid inhibitors, salts and esters thereof; racemic mixtures and resolved isomers thereof; and combinations thereof.

Specific examples of possible herbicides include 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluron, clethodim, clodinafop, clomazone, cyanazine, cycloxydim, cyhalofop, desmedipham, desmetryn, dicamba, diclofop, dimefuron, diuron, dithiopyr, fenoxaprop, fluazifop), fluazifop-P, fluometuron, flufenpyr-ethyl, flumiclorac-pentyl, flumioxazin, fluoroglycofen, fluthiacet-methyl, fomesafe, fomesafen, glyphosate, glufosinate, haloxyfop, hexazinone, imazamox, imazaquin, imazethapyr, ioxynil, isoproturon, isoxaflutole, lactofen, linuron, mecoprop, mecoprop-P, mesotrione, metamitron, metazochlor, methibenzuron, metolachlor (and S-metolachlor), metoxuron, metribuzin, monolinuron, oxadiargyl, oxadiazon, oxyfluorfen, phenmedipham, pretilachlor, profoxydim, prometon, prometryn, propachlor, propanil, propaquizafop, propisochlor, pyraflufen-ethyl, pyrazon, pyrazolynate, pyrazoxyfen, pyridate, quizalofop, quizalofop-P (e.g., quizalofop-ethyl), quizalofop-P-ethyl, clodinafop-propargyl, cyhalofop-butyl, diclofop-methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins as described in U.S. Pat. No. 7,989,393), thenylchlor, tralkoxydim, triclopyr, trietazine, tropramezone, and salts and esters thereof; racemic mixtures and resolved isomers thereof, and combinations thereof.

Commercial products containing each of these herbicides are readily available. Herbicide concentration in the composition will generally correspond to the labeled use rate for a particular herbicide.

Fungicide(s):

In one embodiment, the compositions described herein may further comprise one or more fungicides. Fungicides useful to the compositions described herein may be biological fungicides, chemical fungicides, or combinations thereof. Fungicides may be selected so as to be provide effective control against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). More common fungal pathogens that may be effectively targeted include Pytophthora, Rhizoctonia, Fusarium, Pythium, Phomopsis or Selerotinia and Phakopsora and combinations thereof.

Biological Fungicides:

In embodiments, the biological fungicide can be a bacterium of the genus Actinomycetes, Agrobacterium, Arthrobacter, Alcaligenes, Aureobacterium, Azobacter, Bacillus, Beijerinckia, Brevibacillus, Burkholderia, Chromobacterium, Clostridium, Clavibacter, Comomonas, Corynebacterium, Curtobacterium, Enterobacter, Flavobacterium, Gluconobacter, Hydrogenophage, Klebsiella, Methylobacterium, Paenibacillus, Pasteuria, Phingobacterium, Photorhabdus, Phyllobacterium, Pseudomonas, Rhizobium, Serratia, Stenotrophomonas, Streptomyces, Variovorax, and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Pasteuria penetrans, Pasteuria usage, Pseudomona fluorescens, and combinations thereof.

In embodiments the biological fungicide can be a fungus of the genus Alternaria, Ampelomyces, Aspergillus, Aureobasidium, Beauveria, Candida, Colletotrichum, Coniothyrium, Cryphonectria, Fusarium, Gliocladium, Metarhizium, Metschnikowia, Microdochium, Muscodor, Paecilonyces, Phlebiopsis, Pseudozyma, Pythium, Trichoderma, Typhula, Ulocladium, and Verticilium. In particular embodiments the fungus is Beauveria bassiana, Coniothyrium minitans, Gliocladium virens, Metarhizium anisopliae (also may be referred to in the art as Metarrhizium anisopliae, Metarhizium brunneum, or “green muscadine”), Muscodor albus, Paecilomyces lilacinus, Trichoderma polysporum, and combinations thereof.

Non-limiting examples of biological fungicides that may be suitable for use in the compositions disclosed herein include Ampelomyces quisqualis (e.g., AQ 10® from Intrachem Bio GmbH & Co. KG, Germany), Aspergillus flavus (e.g., AFLAGUARD® from Syngenta, CH), Aureobasidium pullulans (e.g., BOTECTOR® from bio-ferm GmbH, Germany), Bacillus pumilus, Bacillus pumilus isolate AQ717, NRRL B-21662 (from Fa. AgraQuest Inc., USA), Bacillus pumilus isolate NRRL B-30087 (from Fa. AgraQuest Inc., USA), Bacillus sp., isolate AQ175, ATCC 55608 (from Fa. AgraQuest Inc., USA), Bacillus sp., isolate AQ177, ATCC 55609) (from Fa. AgraQuest Inc., USA), Bacillus subtilis, Bacillus subtilis isolate AQ713, NRRL B-21661 (in RHAPSODY®, SERENADE® MAX and SERENADE® ASO) (from Fa. AgraQuest Inc., USA), Bacillus subtilis isolate AQ743, NRRL B-21665 (from Fa. AgraQuest Inc., USA), Bacillus amyloliquefaciens, Bacillus amyloliquefaciens FZB24 (e.g., deposited as isolates NRRL B-50304 and NRRL B-50349 TAEGRO® from Novozymes Biologicals, Inc., USA), Bacillus amyloliquefaciens TJ1000 (i.e., also known as 1 BE, isolate ATCC BAA-390), Bacillus thuringiensis, Bacillus thuringiensis isolate AQ52, NRRL B-21619 (from Fa. AgraQuest Inc., USA), Candida oleophila, Candida oleophila 1-82 (e.g., ASPIRE® from Ecogen Inc., USA), Candida saitoana (e.g., BIOCURE® in mixture with lysozyme) and BIOCOAT® from Micro Flo Company, USA (BASF SE) and Arysta), Clonostachys rosea f. catenulata, also named Gliocladium catenulatum (e.g., isolate J1446: PRESTOP® from Verdera, Finland), Coniothyrium minitans (e.g., CONTANS® from Prophyta, Germany), Cryphonectria parasitica (e.g., Endothia parasitica from CNICM, France), Cryptococcus albidus (e.g., YIELD PLUS® from Anchor Bio-Technologies, South Africa), Fusarium oxysporum (e.g., BIOFOX® from S.I.A.P.A., Italy, FUSACLEAN® from Natural Plant Protection, France), Metschnikowia fructicola (e.g., SHEMER® from Agrogreen, Israel), Microdochium dimerum (e.g., ANTIBOT® from Agrauxine, France), Muscodor albus, Muscador albus isolate NRRL 30547 (from Fa. AgraQuest Inc., USA), Muscador roseus, Muscador roseus isolate NRRL 30548 (from Fa. AgraQuest Inc., USA), Phlebiopsis gigantea (e.g., ROTSOP® from Verdera, Finland), Pseudozyma flocculosa (e.g., SPORODEX® from Plant Products Co. Ltd., Canada), Pythium oligandrum, Pythium oligandrum DV74 (e.g., POLYVERSUM® from Remeslo SSRO, Biopreparaty, Czech Rep.), Talaromyces flavus, Talaromyces flavus V117b (e.g., PROTUS® from Prophyta, Germany), Trichoderma asperellum, Trichoderma asperellum SKT-1 (e.g., ECO-HOPE® from Kumiai Chemical Industry Co., Ltd., Japan), Trichoderma atroviride, Trichoderma atroviride LC52 (e.g., SENTINEL® from Agrimm Technologies Ltd, NZ), Trichoderma harzianum Trichoderma harzianum T-22 (e.g., PLANTSHIELD® der Firma BioWorks Inc., USA), Trichoderma harzianum TH-35 (e.g., ROOT PRO® from Mycontrol Ltd., Israel), Trichoderma harzianum T-39 (e.g., TRICHODEX® and TRICHODERMA 2000® from Mycontrol Ltd., Israel and Makhteshim Ltd., Israel), Trichoderma harzianum ICC012, Trichoderma harzianum and Trichoderma viride (e.g., TRICHOPEL from Agrimm Technologies Ltd, NZ), Trichoderma harzianum ICC012 and Trichoderma viride ICC080 (e.g., REMEDIER® WP from Isagro Ricerca, Italy), Trichoderma polysporum and Trichoderma harzianum (e.g., BINAB® from BINAB Bio-Innovation AB, Sweden), Trichoderma stromaticum (e.g., TRICOVAB® from C.E.P.L.A.C., Brazil), Trichoderma virens, Trichoderma virens GL-21 (e.g., SOILGARD® from Certis LLC, USA), Trichoderma virens G1-3 (e.g., ATCC 58678, from Novozymes BioAg, Inc.), Trichoderma virens G1-21 (commercially available from Thermo Trilogy Corporation), Trichoderma virens and Bacillus amyloliquefaciens, Trichoderma virens G1-3 and Bacillus amyloliquefaciens FZB24, Trichoderma virens G1-3 and Bacillus amyloliquefaciens isolate NRRL B-50349, Trichoderma virens G1-3 and Bacillus amyloliquefaciens TJ 1000, Trichoderma virens G1-21 and Bacillus amyloliquefaciens FZB24, Trichoderma virens G1-21 and Bacillus amyloliquefaciens isolate NRRL B-50349, Trichoderma virens G1-21 and Bacillus amyloliquefaciens TJ1000, Trichoderma viride (e.g., TRIECO® from Ecosense Labs. (India) Pvt. Ltd., Indien, BIO-CURE® F from T. Stanes & Co. Ltd., Indien), Trichoderma viride TV1 (e.g., Trichoderma viride TV1 from Agribiotec srl, Italy), Trichoderma viride ICC080, Streptomyces sp. isolate NRRL No. B-30145 (from Fa. AgraQuest Inc., USA), Streptomyces sp. isolate M1064 (from Fa. AgraQuest Inc., USA), Streptomyces galbus, Streptomyces galbus isolate NRRL 30232 (from Fa. AgraQuest Inc., USA), Streptomyces lydicus, Streptomyces lydicus WYEC 108 (e.g., isolate ATCC 55445 in ACTINOVATE®, ACTINOVATE AG®, ACTINOVATE STP®, ACTINO-IRON®, ACTINOVATE L&G®, and ACTINOGROW® from Idaho Research Foundation, USA), Streptomyces violaceusniger, Streptomyces violaceusniger YCED 9 (e.g., isolate ATCC 55660 in DE-THATCH-9®, DECOMP-9®, and THATCH CONTROL® from Idaho Research Foundation, USA), Streptomyces WYE 53 (e.g., isolate ATCC 55750 in DE-THATCH-9®, DECOMP-9®, and THATCH CONTROL® from Idaho Research Foundation, USA) and Ulocladium oudemansii, Ulocladium oudemansii HRU3 (e.g., BOTRY-ZEN® from Botry-Zen Ltd, NZ).

In further embodiments the biological fungicide can be plant growth activators or plant defense agents including, but not limited to harpin, Reynoutria sachlinensis (e.g., REGALIA® (from Marrone Biolnnovations, USA).

Chemical Fungicides

In certain embodiments, the fungicide is a chemical fungicide. Representative examples of useful chemical fungicides that may be suitable for use in the present disclosure include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles:

A) Strobilurins:

azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester, and 2-(2-(3-(2,6-dichlorophenyl)-1-methyl-allylideneaminooxymethyl)-phenyl)-2-methoxyimino-N-methyl-acetamide;

B) Carboxamides:

carboxanilides: benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyrazole-4-carboxamide, and N-(2-(1,3,3-trimethylbutyl)-phenyl)-1,3-dimethyl-5-fluoro-1H-pyrazole-4-carboxamide;

carboxylic morpholides: dimethomorph, flumorph, pyrimorph;

benzoic acid amides: flumetover, fluopicolide, fluopyram, zoxamide;

other carboxamides: carpropamid, dicyclomet, mandiproamid, oxytetracyclin, silthiofam, and N-(6-methoxy-pyridin-3-yl) cyclopropanecarboxylic acid amide;

C) Azoles:

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole;

imidazoles: cyazofamid, imazalil, pefurazoate, prochloraz, triflumizol;

D) Heterocyclic Compounds:

pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine;

pyrimidines: bupirimate, cyprodinil, diflumetorim, fenarimol, ferimzone, mepanipyrim, nitrapyrin, nuarimol, pyrimethanil;

piperazines: triforine;

pyrroles: fenpiclonil, fludioxonil;

morpholines: aldimorph, dodemorph, dodemorph-acetate, fenpropimorph, tridemorph;

piperidines: fenpropidin;

dicarboximides: fluoroimid, iprodione, procymidone, vinclozolin;

non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;

others: acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, and 5-chloro-7-(4-methylpiperidin-1-yl)-6-(2,4,6-trifluorophenyl)-[1,2,4]triazolo-[1,5-a]pyrimidine;

E) Benzimidazoles: Carbendazim.

F) Other Active Substances:

guanidines: guanidine, dodine, dodine free base, guazatine, guazatine-acetate, iminoctadine, iminoctadine-triacetate, iminoctadine-tris(albesilate);

antibiotics: kasugamycin, kasugamycin hydrochloride-hydrate, streptomycin, polyoxine, validamycin A;

nitrophenyl derivates: binapacryl, dicloran, dinobuton, dinocap, nitrothal-isopropyl, tecnazen,

organometal compounds: fentin salts, such as fentin-acetate, fentin chloride, or fentin hydroxide;

sulfur-containing heterocyclyl compounds: dithianon, isoprothiolane;

organophosphorus compounds: edifenphos, fosetyl, fosetyl-aluminum, iprobenfos, phosphorus acid and its salts, pyrazophos, tolclofos-methyl;

organochlorine compounds: chlorothalonil, dichlofluanid, dichlorophen, flusulfamide, hexachlorobenzene, pencycuron, pentachlorphenole and its salts, phthalide, quintozene, thiophanate-methyl, thiophanate, tolylfluanid, N-(4-chloro-2-nitro-phenyl)-N-ethyl-4-methyl-benzenesulfonamide;

inorganic active substances: Bordeaux mixture, copper acetate, copper hydroxide, copper oxychloride, basic copper sulfate, and sulfur.

Commercial fungicides are most suitably used in accordance with the manufacturer's instructions at the recommended concentrations.

Insecticide(s), Acaricide(s), Nematicide(s):

In one embodiment, the compositions described herein may further comprise one or more insecticides, acaricides, nematicides, or combinations thereof. Insecticides, acaricides, and/or nematicides useful to the compositions described herein will suitably exhibit activity against a broad range of nematodes, insects, and acarids. The pesticides described herein may be chemical pesticides microbial pesticides (e.g., biological solutions, such as fungal pesticides, bacterial pesticides, etc.), or combinations thereof.

Chemical Insecticides, Acaricides, Nematicides:

Non-limiting examples of chemical insecticides, acaricides, and nematicides that may be useful to the compositions disclosed herein include carbamates, diamides, macrocyclic lactones, neonicotinoids, organophosphates, phenylpyrazoles, pyrethrins, spinosyns, synthetic pyrethroids, tetronic acids, and tetramic acids.

In particular embodiments useful chemical insecticides, acaricides, and nematicides include acrinathrin, alpha-cypermethrin, betacyfluthrin, cyhalothrin, cypermethrin, deltamethrin, csfenvalcrate, etofenprox, fenpropathrin, fenvalerate, flucythrinat, fosthiazate, lambda-cyhalothrin, gamma-cyhalothrin, permethrin, tau-fluvalinate, transfluthrin, zeta-cypermethrin, cyfluthrin, bifenthrin, tefluthrin, eflusilanat, fubfenprox, pyrethrin, resmethrin, imidacloprid, acetamiprid, thiamethoxam, nitenpyram, thiacloprid, dinotefuran, clothianidin, imidaclothiz, chlorfluazuron, diflubenzuron, lufenuron, teflubenzuron, triflumuron, novaluron, flufenoxuron, hexaflumuron, bistrifluoron, noviflumuron, buprofezin, cyromazine, methoxyfenozide, tebufenozide, halofenozide, chromafenozide, endosulfan, fipronil, ethiprole, pyrafluprole, pyriprole, flubendiamide, chlorantraniliprole (e.g., Rynaxypyr), cyazypyr, emamectin, emamectin benzoate, abamectin, ivermectin, milbemectin, lepimectin, tebufenpyrad, fenpyroximate, pyridaben, fenazaquin, pyrimidifen, tolfenpyrad, dicofol, cyenopyrafen, cyflumetofen, acequinocyl, fluacrypyrin, bifenazate, diafenthiuron, etoxazole, clofentezine, spinosad, triarathen, tetradifon, propargite, hexythiazox, bromopropylate, chinomethionat, amitraz, pyrifluquinazon, pymetrozine, flonicamid, pyriproxyfen, diofenolan, chlorfenapyr, metaflumizone, indoxacarb, chlorpyrifos, spirodiclofen, spiromesifen, spirotetramat, pyridalyl, spinctoram, acephate, triazophos, profenofos, oxamyl, spinetoram, fenamiphos, fenamipclothiahos, 4-{[(6-chloropyrid-3-yl)methyl](2,2-difluoroethyl)amino}furan-2(5H)-one, cadusaphos, carbaryl, carbofuran, ethoprophos, thiodicarb, aldicarb, aldoxycarb, metamidophos, methiocarb, sulfoxaflor, cyantraniliprole, tioxazofen, and combinations thereof.

Microbial Insecticides, Acaricides, and Nematicides:

A) Fungal Insecticides, Acaricides, and Nematicides:

In a particular embodiment, the microbial insecticides, acaricides, and nematicides are one or more fungal insecticides, acaricides, and nematicides. Non-limiting examples of fungal insecticides, acaricides, and nematicides that may be used in the compositions disclosed herein are described in McCoy, C. W., Samson, R. A., and Coucias, D. G. “Entomogenous fungi. In “CRC Handbook of Natural Pesticides. Microbial Pesticides, Part A. Entomogenous Protozoa and Fungi.” (C. M. Inoffo, ed.), (1988): Vol. 5, 151-236; Samson, R. A., Evans, H. C., and Latgé, J. P. “Atlas of Entomopathogenic Fungi.” (Springer-Verlag, Berlin) (1988); and deFaria, M. R. and Wraight, S. P. “Mycoinsecticides and Mycoacaricides: A comprehensive list with worldwide coverage and international classification of formulation types.” Biol. Control (2007), doi: 10.1016/j.biocontrol.2007.08.001.

In embodiments, the fungal insecticides, acaricides, and nematicides can be a fungus of the genus Aegerita, Akanthomyces, Alternaria, Arthrobotrys, Aschersonia, Ascophaera, Aspergillus, Beauveria, Blastodendrion, Calonectria, Coelemomyces, Coelomycidium, Conidiobolus, Cordyceps, Couchia, Culicinomyces, Dactylaria, Engyodontium, Entomophaga, Entomophthora, Erynia, Filariomyces, Filobasidiella, Fusarium, Gibellula, Harposporium, Hesperomyces, Hirsutella, Hymenostilbe, Hypocrella, Isaria, Lecanicillium, Lagenidium, Leptolegnia, Massospora, Metarhizium, Meristacrum, Metschnikowia, Monacrosporium, Mycoderma, Myiophagus, Myriangium, Myrothecium, Nectria, Nematoctonus, Neozygites, Nomuraea, Paecilomyces, Pandora, Paraisaria, Pasteuria, Pleurodesmospora, Pochonia, Podonectria, Polycephalomyces, Pseudogibellula, Septobasidium, Sorosporella, Sporodiniella, Stillbella, Tetranacrium, Tilachlidium, Tolypocladium, Torrubiella, Trenomyces, Trichoderma, Uredinella, Verticillium, Zoophthora, and combinations thereof.

Non-limiting examples of particular species that may be useful as a fungal insecticide, acaricide, and nematicide in the compositions described herein include Alternaria cassia, Arthrobotrys dactyloides, Arthrobotrys oligospora, Arthrobotrys superb, Arthrobotrys dactyloides, Aspergillus parasiticus, Beauveria bassiana, Beauveria bassiana isolate ATCC-74040, Beauveria bassiana isolate ATCC-74250, Dactylaria candida, Fusarium lateritum, Fusarium solani, Harposporium anguillulae, Hirsutella rhossiliensis, Hirsutella minnesotensis, Lecanicillium lecanii, Metarhizium anisopliae (also may be referred to in the art as Metarrhizium anisopliae, Metarhizium brunneum, or “green muscadine”), Metarhizium anisopliae isolate F52 (also known as Metarhizium anisopliae strain 52, Metarhizium anisopliae strain 7, Metarhizium anisopliae strain 43, Metarhizium anisopliae BIO-1020, TAE-001 and deposited as DSM 3884, DSM 3885, ATCC 90448, SD 170, and ARSEF 7711) (available from Novozymes Biologicals, Inc., USA)), Monacrosporium cionopagum, Nematoctonus geogenius, Nematoctonus leiosporus, Meristacrum asterospermum, Myrothecium verrucaria, Paecilomyces fumosoroseus, Paecilomyces fumosoroseus FE991 (in NOFLY® from FuturEco BioScience S.L., Barcelona, Spain), Paecilomyces lilacinus, Pasteuria penetrans, Pasteuria usage, Pochonia chlamydopora, Trichoderma hamatum, Trichoderma harzianum, Trichoderma virens, Verticillium chlamydosporum, Verticillium lecanii, and combinations thereof.

B) Bacterial Insecticides, Acaricides, and Nematicides:

In a particular embodiment, the microbial insecticides, acaricides, and nematicides are one or more bacterial insecticides, acaricides, and nematicides.

In embodiments, the bacterial insecticides, acaricides, and nematicides can be a bacterium of the genus Actinomycetes Agrobacterium, Arthrobacter, Alcaligenes, Aureobacterium, Azobacter, Bacillus, Beijerinckia, Burkholderia, Chromobacterium, Clavibacter, Clostridium, Comomonas, Corynebacterium, Curtobacterium, Desulforibtio, Enterobacter, Flavobacterium, Gluconobacter, Hydrogenophage, Klebsiella, Methylobacterium, Paenibacillus, Phyllobacterium, Phingobacterium, Photorhabdus, Pseudomonas, Rhodococcus, Serratia, Stenotrotrophomonas, Streptomyces, Xenorhadbus, Variovorax, and combinations thereof.

Non-limiting examples of particular species that may be useful as bacterial insecticides, acaricides, and nematicides in the compositions described herein include Bacillus firmus, Bacillus firmus isolate 1-1582 (in BioNeem, Votivo), Bacillus mycoides, Bacillus mycoides isolate AQ726, NRRL B-21664, Burkholderia sp., Burkholderia sp. nov. rinojensis, Burkholderia sp. A396 sp. nov. rinojensis, NRRL B-50319, Chromobacterium subtsugae, Chromobacterium subtsugae sp. nov., Chromobacterium subtsugae sp. nov. isolate NRRL B-30655, Chromobacterium vaccinii, Chromobacterium vaccinii isolate NRRL B-50880, Chromobacterium violaceum, Flavobacterium sp., Flavobacterium sp. isolate H492, NRRL B-50584, Streptomyces lydicus, Streptomyces violaceusniger, and combinations thereof.

Commercial insecticides, acaricides, and nematicides are most suitably used in accordance with the manufacturer's instructions at the recommended concentrations.

Nutrient(s):

In still another embodiment, the compositions described herein may comprise one or more beneficial nutrients. Non-limiting examples of nutrients for use in the compositions described herein include vitamins, (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.), macrominerals (e.g., phosphorous, calcium, magnesium, potassium, sodium, iron, etc.), trace minerals (e.g., boron, cobalt, chloride, chromium, copper, fluoride, iodine, iron, manganese, molybdenum, selenium, zinc, etc.), organic acids (e.g., acetic acid, citric acid, lactic acid, malic acid, taurine, etc.), and combinations thereof. In a particular embodiment, the compositions may comprise phosphorous, boron, chlorine, copper, iron, manganese, molybdenum, zinc or combinations thereof.

In certain embodiments, where the compositions described herein may comprise phosphorous, it is envisioned that any suitable source of phosphorous may be provided. In one embodiment, the phosphorus may be derived from a source. In another embodiment, suitable sources of phosphorous include phosphorous sources capable of solubilization by one or more microorganisms (e.g., Penicillium bilaiae, etc.).

In one embodiment, the phosphorus may be derived from a rock phosphate source. In another embodiment the phosphorous may be derived from fertilizers comprising one or more phosphorous sources. Commercially available manufactured phosphate fertilizers are of many types. Some common ones are those containing rock phosphate, monoammonium phosphate, diammonium phosphate, monocalcium phosphate, super phosphate, triple super phosphate, and/or ammonium polyphosphate. All of these fertilizers are produced by chemical processing of insoluble natural rock phosphates in large scale fertilizer-manufacturing facilities and the product is expensive. Accordingly, it is possible to reduce the amount of these fertilizers applied to the soil while still maintaining the same amount of phosphorus uptake from the soil.

In still another embodiment, the phosphorous may be derived from an organic phosphorous source. In a further particular embodiment, the source of phosphorus may include an organic fertilizer. An organic fertilizer refers to a soil amendment derived from natural sources that guarantees, at least, the minimum percentages of nitrogen, phosphate, and potash. Non-limiting examples of organic fertilizers include plant and animal by-products, rock powders, seaweed, inoculants, and conditioners. These are often available at garden centers and through horticultural supply companies. In particular the organic source of phosphorus is from bone meal, meat meal, animal manure, compost, sewage sludge, or guano, or combinations thereof.

In still another embodiment, the phosphorous may be derived from a combination of phosphorous sources including, but not limited to, rock phosphate, fertilizers comprising one or more phosphorous sources (e.g., monoammonium phosphate, diammonium phosphate, monocalcium phosphate, super phosphate, triple super phosphate, ammonium polyphosphate, etc.) one or more organic phosphorous sources, and combinations thereof.

Biostimulant(s):

In one embodiment, the compositions described herein may comprise one or more beneficial biostimulants. Biostimulants may enhance metabolic or physiological processes such as respiration, photosynthesis, nucleic acid uptake, ion uptake, nutrient delivery, or a combination thereof. Non-limiting examples of biostimulants include seaweed extracts (e.g., ascophyllum nodosum), humic acids (e.g., potassium humate), fulvic acids, myo-inositol, glycine, and combinations thereof. In another embodiment, the compositions comprise seaweed extracts, humic acids, fulvic acids, myo-inositol, glycine, and combinations thereof.

Polymer(s):

In one embodiment, the compositions described herein may further comprise one or more polymers. Non-limiting uses of polymers in the agricultural industry include agrochemical delivery, heavy metal removal, water retention and/or water delivery, and combinations thereof. Pouci, et al., Am. J. Agri. & Biol. Sci., 3(1):299-314 (2008). In one embodiment, the one or more polymers is a natural polymer (e.g., agar, starch, alginate, pectin, cellulose, etc.), a synthetic polymer, a biodegradable polymer (e.g., polycaprolactone, polylactide, poly (vinyl alcohol), etc.), or a combination thereof.

For a non-limiting list of polymers useful for the compositions described herein, see Pouci, et al., Am. J. Agri. & Biol. Sci., 3(1):299-314 (2008). In one embodiment, the compositions described herein comprise cellulose, cellulose derivatives, methylcellulose, methylcellulose derivatives, starch, agar, alginate, pectin, polyvinylpyrrolidone, and combinations thereof.

Wetting Agent(s):

In one embodiment, the compositions described herein may further comprise one or more wetting agents. Wetting agents are commonly used on soils, particularly hydrophobic soils, to improve the infiltration and/or penetration of water into a soil. The wetting agent may be an adjuvant, oil, surfactant, buffer, acidifier, or combination thereof. In an embodiment, the wetting agent is a surfactant. In an embodiment, the wetting agent is one or more nonionic surfactants, one or more anionic surfactants, or a combination thereof. In yet another embodiment, the wetting agent is one or more nonionic surfactants.

Surfactants suitable for the compositions described herein are provided in the “Surfactants” section.

Surfactant(s):

Surfactants suitable for the compositions described herein may be non-ionic surfactants (e.g., semi-polar and/or anionic and/or cationic and/or zwitterionic). The surfactants can wet and emulsify soil(s) and/or dirt(s). It is envisioned that the surfactants used in described composition have low toxicity for any microorganisms contained within the formulation. It is further envisioned that the surfactants used in the described composition have a low phytotoxicity (i.e., the degree of toxicity a substance or combination of substances has on a plant). A single surfactant or a blend of several surfactants can be used.

Anionic Surfactants

Anionic surfactants or mixtures of anionic and nonionic surfactants may also be used in the compositions. Anionic surfactants are surfactants having a hydrophilic moiety in an anionic or negatively charged state in aqueous solution. The compositions described herein may comprise one or more anionic surfactants. The anionic surfactant(s) may be either water soluble anionic surfactants, water insoluble anionic surfactants, or a combination of water soluble anionic surfactants and water insoluble anionic surfactants. Non-limiting examples of anionic surfactants include sulfonic acids, sulfuric acid esters, carboxylic acids, and salts thereof. Non-limiting examples of water soluble anionic surfactants include alkyl sulfates, alkyl ether sulfates, alkyl amido ether sulfates, alkyl aryl polyether sulfates, alkyl aryl sulfates, alkyl aryl sulfonates, monoglyceride sulfates, alkyl sulfonates, alkyl amide sulfonates, alkyl aryl sulfonates, benzene sulfonates, toluene sulfonates, xylene sulfonates, cumene sulfonates, alkyl benzene sulfonates, alkyl diphenyloxide sulfonate, alpha-olefin sulfonates, alkyl naphthalene sulfonates, paraffin sulfonates, lignin sulfonates, alkyl sulfosuccinates, ethoxylated sulfosuccinates, alkyl ether sulfosuccinates, alkylamide sulfosuccinates, alkyl sulfosuccinamate, alkyl sulfoacetates, alkyl phosphates, phosphate ester, alkyl ether phosphates, acyl sarconsinates, acyl isethionates, N-acyl taurates, N-acyl-N-alkyltaurates, alkyl carboxylates, or a combination thereof.

Nonionic Surfactants

Nonionic surfactants are surfactants having no electrical charge when dissolved or dispersed in an aqueous medium. In at least one embodiment of the composition described herein, one or more nonionic surfactants are used as they provide the desired wetting and emulsification actions and do not significantly inhibit spore stability and activity. The nonionic surfactant(s) may be either water soluble nonionic surfactants, water insoluble nonionic surfactants, or a combination of water soluble nonionic surfactants and water insoluble nonionic surfactants.

Water Insoluble Nonionic Surfactants

Non-limiting examples of water insoluble nonionic surfactants include alkyl and aryl: glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, or combinations thereof. Also included are EO/PO block copolymers (EO is ethylene oxide, PO is propylene oxide), EO polymers and copolymers, polyamines, and polyvinylpynolidones.

Water Soluble Nonionic Surfactants

Non-limiting examples of water soluble nonionic surfactants include sorbitan fatty acid alcohol ethoxylates and sorbitan fatty acid ester ethoxylates.

Combination of Nonionic Surfactants

In one embodiment, the compositions described herein comprise at least one or more nonionic surfactants. In one embodiment, the compositions comprise at least one water insoluble nonionic surfactant and at least one water soluble nonionic surfactant. In still another embodiment, the compositions comprise a combination of nonionic surfactants having hydrocarbon chains of substantially the same length.

Other Surfactants

In another embodiment, the compositions described herein may also comprise organosilicone surfactants, silicone-based antifoams used as surfactants in silicone-based and mineral-oil based antifoams. In yet another embodiment, the compositions described herein may also comprise alkali metal salts of fatty acids (e.g., water soluble alkali metal salts of fatty acids and/or water insoluble alkali metal salts of fatty acids).

Anti-Freezing Agent(s):

In one embodiment, the compositions described herein may further comprise one or more anti-freezing agents. Non-limiting examples of anti-freezing agents include ethylene glycol, propylene glycol, urea, glycerin, and combinations thereof.

### Methods

In another aspect, methods of using the compositions described herein are disclosed. In a particular embodiment, the method includes treating a plant or plant part comprising contacting a plant or plant part with one or more microbial spores and one or more germinants. In one embodiment, the plant or plant part is contacted by the one or more microbial spores sequentially (i.e., before or after) with the one or more germinants. In another embodiment, the plant or plant part is contacted by the one or more microbial spores simultaneously (i.e., at or about the same time) with the one or more germinants. In a particular embodiment the method includes treating a plant or plant part comprising contacting a plant or plant part with one or more compositions described herein.

The applying step can be performed by any method known in the art (including both foliar and non-foliar applications). Non-limiting examples of applying to the plant or plant part include spraying a plant or plant part, drenching a plant or plant part, dripping on a plant or plant part, dusting a plant or plant part, and/or coating a seed. In a more particular embodiment, the applying step is repeated (e.g., more than once, as in the contacting step is repeated twice, three times, four times, five times, six times, seven times, eight times, nine times, ten times, etc.).

In a particular embodiment the contacting step comprises foliarly applying to a plant or plant part (i.e., application to the plant by spraying, e.g., via foliar spray, a predosage device, a knapsack sprayer, a spray tank or a spray plane) one or more microbial spores and one or more germinants. In still yet a more particular embodiment, the contacting step comprises foliarly applying one or more compositions described herein to plant foliage.

In another embodiment, the method further comprises applying to the plant or plant part one or more agriculturally beneficial ingredients described herein. In one embodiment the one or more agriculturally beneficial ingredients are applied simultaneously or sequentially with the one or more microbial spores. In another embodiment the one or more agriculturally beneficial ingredients are applied simultaneously or sequentially with the one or more germinants.

Application of the one or more agriculturally beneficial ingredients may also be applied to the plant or plant parts as part of a composition described herein or applied independently from the one or more compositions described herein. In one embodiment, the one or more agriculturally beneficial ingredients are applied to the plant or plant parts as part of one or more of the compositions described herein. In another embodiment, the one or more agriculturally beneficial ingredients are applied to the plant or plant parts independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In a yet another aspect, a method for inducing the germination of a microbial spore is described herein. In one embodiment, the method comprises inducing the germination of a microorganism comprising foliarly applying one or more microbial spores and one or more germinants to a plant or plant part, wherein upon foliar application of the one or more microbial spores and the one or more germinants to a plant or plant part, the one or more microbial spores exhibit increased germination on the plant or plant part in the presence of the one or more germinants compared to the foliar application of one or more microbial spores alone (i.e., without one or more germinants) on a plant or plant part. As used herein, the terms “increased germination” “enhanced germination” and/or variations thereof, is intended to mean an increase in the proportion of applied spores that germinate in the presence of a germinant when compared to the proportion of applied spores that germinate in the absence of a germinant; the increase in speed by which applied spores germinate in the presence of a germinant when compared to the speed by which applied spores germinate in the absence of a germinant, or combinations thereof. In a more particular embodiment, the method for inducing germination of a microbial spore comprises foliarly applying one or more bacterial spores and one or more germinants to plant foliage. In still another embodiment, the method for inducing germination of a microbial spore comprises foliarly applying one or more compositions described herein.

The method may further comprise subjecting the plant or plant part to one or more agriculturally beneficial ingredients, applied simultaneously or sequentially with the one or more microbial spores or one or more germinants. In one embodiment the one or more agriculturally beneficial ingredients are applied simultaneously or sequentially with the one or more microbial spores. In another embodiment the one or more agriculturally beneficial ingredients are applied simultaneously or sequentially with the one or more germinants. Application of the one or more agriculturally beneficial ingredients may also be applied to the plant or plant parts as part of a composition described herein or applied independently from the one or more compositions described herein. In one embodiment, the one or more agriculturally beneficial ingredients are applied to the plant or plant parts as part of one or more of the compositions described herein. In another embodiment, the one or more agriculturally beneficial ingredients are applied to the plant or plant parts independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In another aspect, a method for treating soil is described herein. In one embodiment, the method comprises contacting a soil with one or more microbial spores and one or more germinants. In another embodiment, the method comprises contacting a soil with one or more microbial spores and one or more germinants, and growing a plant or plant part in the treated soil. In still yet another embodiment, the method comprises contacting a soil with one or more of the compositions described herein, and growing a plant or plant part in the treated soil.

In an embodiment, the contacting step can be performed by any method known in the art. Non-limiting examples of contacting the soil include spraying the soil, drenching the soil, dripping onto the soil, and/or dusting the soil. In one embodiment, the contacting step is repeated (e.g., more than once, as in the contacting step is repeated twice, three times, four times, five times, six times, seven times, eight times, nine times, ten times, etc.). In one embodiment, the contacting step comprises contacting the soil with one or more microbial spores sequentially with one or more germinants. In another embodiment, the contacting step comprises contacting the soil with one or more microbial spores simultaneously with one or more germinants. In a particular embodiment, the contacting step comprises introducing one or more of the compositions described herein to the soil.

The contacting step can occur at any time during the growth of the plant or plant part. In one embodiment, the contacting step occurs before the plant or plant part begins to grow. In another embodiment, the contacting step occurs after the plant or plant part has started to grow.

In another embodiment, the method further comprises the step of planting a plant or plant part. The planting step can occur before, after or during the contacting step. In one embodiment, the planting step occurs before the contacting step. In another embodiment, the planting step occurs during the contacting step (e.g., the planting step occurs simultaneously with the contacting step, the planting step occurs substantially simultaneous with the contacting step, etc.). In still another embodiment, the planting step occurs after the contacting step.

The method may further comprise subjecting the soil to one or more agriculturally beneficial ingredients, applied simultaneously or sequentially with the one or more microbial spores or one or more germinants. In one embodiment the one or more agriculturally beneficial ingredients are applied simultaneously or sequentially with the one or more microbial spores. In another embodiment the one or more agriculturally beneficial ingredients are applied simultaneously or sequentially with the one or more germinants. Application of the one or more agriculturally beneficial ingredients may also be applied to the soil as part of a composition described herein or applied independently from the one or more compositions described herein. In one embodiment, the one or more agriculturally beneficial ingredients are applied to the soil as of one or more of the compositions described herein. In another embodiment, the one or more agriculturally beneficial ingredients are applied to the soil independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In one embodiment, the step of subjecting the soil to one or more agriculturally beneficial ingredients occurs sequentially or simultaneously with the contacting step. In one embodiment, the step of subjecting the soil to one or more agriculturally beneficial ingredients as described herein occurs before the contacting step. In another embodiment, the step of subjecting the soil to one or more agriculturally beneficial ingredients as described herein occurs during the contacting step. In still another embodiment, the step of subjecting the soil to one or more agriculturally beneficial ingredients as described herein occurs after the contacting step. In yet another embodiment, the step of subjecting the soil to one or more agriculturally beneficial ingredients as described herein occurs simultaneously with the contacting step (e.g., contacting the soil with one or more of the compositions described herein, etc.).

The methods described herein are applicable to both leguminous and non-leguminous plants or plant parts. In a particular embodiment the plants or plant parts are selected from the group consisting of alfalfa, rice, wheat, barley, rye, oat, cotton, canola, sunflower, peanut, corn, potato, sweet potato, bean, pea, chickpeas, lentil, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

### Seed Coatings

In another aspect, seeds may be treated with one or more microbial spores and one or more germinants. In a particular embodiment, seeds may be treated with one or more of the compositions described herein. Seed coating methods are well known in the art. In one embodiment the seeds are coated with a dry composition as described herein. In another embodiment seeds are coated with a liquid composition as described herein. In yet another embodiment, the compositions described herein are formulated (e.g., mixed, added, etc.) with a seed treatment mixture.

Coating of the seed may occur in several ways but preferably via spraying or dripping. Spray and drip treatment may be conducted by formulating compositions described herein and spraying or dripping the composition(s) onto a seed(s) via a continuous treating system (which is calibrated to apply treatment at a predefined rate in proportion to the continuous flow of seed), such as a drum-type of treater. Batch systems, in which a predetermined batch size of seed and composition(s) as described herein are delivered into a mixer, may also be employed. Systems and apparati for performing these processes are commercially available from numerous suppliers, e.g., Bayer CropScience (Gustafson).

In another embodiment, the treatment entails coating seeds. One such process involves coating the inside wall of a round container with the composition(s) described herein, adding seeds, then rotating the container to cause the seeds to contact the wall and the composition(s), a process known in the art as “container coating”. Seeds can be coated by combinations of coating methods. Soaking typically entails using liquid forms of the compositions described. For example, seeds can be soaked for about 1 minute to about 24 hours (e.g., for at least 1 min, 5 min, 10 min, 20 min, 40 min, 80 min, 3 hr, 6 hr, 12 hr, 24 hr).

In certain embodiments, a seed(s) coated with one or more of the compositions described herein will comprise 1×10¹-1×10⁸, more preferably 1×10²-1×10⁶ colony forming units of one or more microbial strains per seed.

The embodiments of the disclosure are further defined by the following numbered paragraphs:

1. A composition comprising:
a. a carrier;
b. one or more microbial spores; and
c. one or more germinants,
   wherein the composition is a substantially dry composition.

2. The composition of paragraph 1, wherein the germinant is selected from the group consisting of lactate, lactose, bicarbonate, fructose, glucose, mannose, galactose, alanine, asparagine, cysteine, glutamine, norvatine, serine, threonine, valine, glycine, inosine, taurocholate, and combinations thereof.

3. The composition of paragraph 1, wherein the germinant is a combination of L-asparagine, glucose, fructose, and potassium ion (AGFK).

4. The composition of paragraph 1, wherein the one or more microbial spores is one or more bacterial spores.

5. The composition of paragraph 4, wherein the one or more bacterial spores are selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribaciflus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

6. The composition of paragraph 4, wherein the one or more bacterial spores are one or more Bacillus spores.

7. The composition of paragraph 6, wherein the one or more Bacillus spores are selected from the group consisting of Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, and combinations thereof.

8. The composition of paragraph 6, wherein the one or more Bacillus spores are selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.

9. The composition of paragraph 1, wherein the composition further comprises one or more agriculturally beneficial ingredients.

10. The composition of paragraph 9, wherein the one or more agriculturally beneficial ingredients are one or more biologically active ingredients.

11. The composition of paragraph 10, wherein the one or more biologically active ingredients are selected from the group consisting of one or more plant signal molecules, one or more beneficial microorganisms, and combinations thereof.

12. The composition of paragraph 1, wherein the composition further comprises one or more plant signal molecules.

13. The composition of paragraph 12, wherein the one or more plant signal molecules is a lipo-chitooligosaccharide (LCO).

14. The composition of paragraph 13, wherein the LCO is synthetic.

15. The composition of paragraph 13, wherein the LCO is recombinant.

16. The composition of paragraph 13, wherein the LCO is naturally occurring.

17. The composition of paragraph 13, wherein the LCO is obtained from a species of Rhizobia selected from Rhizobium spp., Bradyrhizobium spp., Sinorhizobium spp., Azorhizobium spp., or a combination thereof.

18. The composition of paragraph 13, wherein the LCO is obtained from Bradyrhizobium japonicum.

19. The composition of paragraph 13, wherein the LCO is obtained from an arbuscular mycorrhizal fungus.

20. The composition of paragraph 12, wherein the plant signal molecule is a chitinous compound.

21. The composition of paragraph 20, wherein the chitinous compound is a chito-oligomer (CO).

22. The composition of paragraph 21, wherein the CO is synthetic.

23. The composition of paragraph 21, wherein the CO is recombinant.

24. The composition of paragraph 21, wherein the CO is naturally occurring.

25. The composition of paragraph 12, wherein the plant signal molecule is a flavonoid.

26. The composition of paragraph 25, wherein the flavonoid is luteolin, apigenin, tangeritin, quercetin, kaempferol, myricetin, fisetin, isorhamnetin, pachypodol, rhamnazin, hesperetin, naringenin, eriodictyol, homoeriodictyol, taxifolin, dihydroquercetin, dihydrokaempferol, genistein, daidzein, glycitein, catechin, gallocatechin, catechin 3-gallate, gallocatechin 3-gallate, epicatechin, epigallocatechin, epicatechin 3-gallate, epigallocatechin 3-gallate, cyaniding, delphinidin, malvidin, pelargonidin, peonidin, petunidin, or derivatives thereof.

27. The composition of paragraph 12, wherein the plant signal molecule is jasmonic acid or a derivative thereof.

28. The composition of paragraph 12, wherein the plant signal molecule is linoleic acid or a derivative thereof.

29. The composition of paragraph 12, wherein the plant signal molecule is linolenic acid or a derivative thereof.

30. The composition of paragraph 12, wherein the plant signal molecule is a karrikin.

31. A method for treating a plant or plant part comprising contacting a plant or plant part with
a. one or more microbial spores; and
b. one or more germinants.

32. The method of paragraph 31, wherein the contacting comprises foliarly applying to a plant or plant part one or more microbial spores and one or more germinants.

33. The method of paragraph 31, wherein the germinant is selected from the group consisting of lactate, lactose, bicarbonate, fructose, glucose, mannose, galactose, alanine, asparagine, cysteine, glutamine, norvatine, serine, threonine, valine, glycine, inosine, taurocholate, and combinations thereof.

34. The method of paragraph 31, wherein the germinant is a combination of L-asparagine, glucose, fructose, and potassium ion (AGFK).

35. The method of paragraph 31, wherein the one or more microbial spores is one or more bacterial spores.

36. The method of paragraph 35, wherein the one or more bacterial spores are selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridfisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

37. The method of paragraph 35, wherein the one or more bacterial spores are one or more Bacillus spores.

38. The method of paragraph 37, wherein the one or more Bacillus spores are selected from the group consisting of Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, and combinations thereof.

39. The method of paragraph 37, wherein the one or more Bacillus spores are selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.

40. The method of paragraphs 31-39, wherein the one or more microbial spores contact the plant or plant part before, after, or simultaneously with the one or more germinants.

41. The method of paragraphs 31-40, wherein the one or more microbial spores contact the plant or plant part before the one or more germinants contact the plant or plant part.

42. The method of paragraphs 31-40, wherein the one or more microbial spores contact the plant or plant part after the one or more germinants contact the plant or plant part.

43. The method of paragraphs 31-40, wherein the one or more microbial spores and the one or more germinants contact the plant or plant part simultaneously.

44. The method of paragraph 31, wherein the method further comprises applying to the plant or plant part one or more agriculturally beneficial ingredients.

45. The method of paragraph 44, wherein the step of applying to the plant or plant part one or more agriculturally beneficial ingredients occurs simultaneously or sequentially with the step of contacting the plant or plant part one or more microbial spores and one or more germinants.

46. The method of paragraph 45, wherein the agriculturally beneficial ingredient is one or more biologically active ingredients.

47. The method of paragraph 46, wherein the one or more biologically active ingredients are selected from the group consisting of one or more plant signal molecules, one or more beneficial microorganisms, and combinations thereof.

48. The method of paragraph 44, wherein the one or more agriculturally beneficial ingredients are one or more plant signal molecules selected from the group consisting of LCOs, COs, chitinous compounds, flavonoids, jasmonic acid, methyl jasmonate, linoleic acid, linolenic acid, karrikins, and combinations thereof.

49. The method of paragraph 44, wherein the one or more agriculturally beneficial ingredients comprises one or more COs.

50. The method of paragraph 44, wherein the one or more agriculturally beneficial ingredients comprises one or more LCOs.

51. The method of paragraph 44, wherein the one or more agriculturally beneficial ingredients comprises one or more beneficial microorganisms.

52. The method of paragraph 51, wherein the one or more beneficial microorganisms comprise one or more nitrogen fixing microorganisms, one or more phosphate solubilizing microorganisms, one or more mycorrhizal fungi, or combinations thereof.

53. A method for inducing the germination of a microbial spore comprising foliarly applying one or more microbial spores and one or more germinants to a plant or plant part, wherein upon foliar application of the one or more microbial spores and the one or more germinants to a plant or plant part, the one or more microbial spores exhibit increased germination on the plant or plant part in the presence of the one or more germinants compared to the foliar application of one or more microbial spores on a plant or plant part without the one or more germinants.

54. The method of paragraph 53, wherein the germinant is selected from the group consisting of lactate, lactose, bicarbonate, fructose, glucose, mannose, galactose, alanine, asparagine, cysteine, glutamine, norvatine, serine, threonine, valine, glycine, inosine, taurocholate, and combinations thereof.

55. The method of paragraph 53, wherein the germinant is a combination of L-asparagine, D-glucose, D-fructose, and potassium ion (AGFK).

56. The method of paragraph 53, wherein the one or more microbial spores is one or more bacterial spores.

57. The method of paragraph 56, wherein the one or more bacterial spores are selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

58. The method of paragraph 56, wherein the one or more bacterial spores are one or more Bacillus spores.

59. The method of paragraph 58, wherein the one or more Bacillus spores are selected from the group consisting of Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, and combinations thereof.

60. The method of paragraph 58, wherein the one or more Bacillus spores are selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.

61. The method of paragraph 53, wherein the method further comprises subjecting the plant or plant part to one or more agriculturally beneficial ingredients.

62. The method of paragraph 61, wherein the agriculturally beneficial ingredient is one or more biologically active ingredients.

63. The method of paragraph 62, wherein the one or more biologically active ingredients are selected from the group consisting of one or more plant signal molecules, one or more beneficial microorganisms, and combinations thereof.

64. The method of paragraph 61, wherein the one or more agriculturally beneficial ingredients are one or more plant signal molecules selected from the group consisting of LCOs, COs, chitinous compounds, flavonoids, jasmonic acid, methyl jasmonate, linoleic acid, linolenic acid, karrikins, and combinations thereof.

65. The method of paragraph 61, wherein the one or more agriculturally beneficial ingredients comprises one or more COs.

66. The method of paragraph 61, wherein the one or more agriculturally beneficial ingredients comprises one or more LCOs.

67. The method of paragraph 61, wherein the one or more agriculturally beneficial ingredients comprises one or more beneficial microorganisms.

68. The method of paragraph 67, wherein the one or more beneficial microorganisms comprise one or more nitrogen fixing microorganisms, one or more phosphate solubilizing microorganisms, one or more mycorrhizal fungi, or combinations thereof.

69. The method of any of the preceding paragraphs, wherein the plant or plant part is a leguminous plant or plant part.

70. The method of any of the preceding paragraphs, wherein the plant or plant part is a soybean plant or plant part.

71. The method of any of the preceding paragraphs, wherein the plant or plant part is a non-leguminous plant or plant part.

72. The method of any of the preceding paragraphs, wherein the plant or plant part is a corn plant or plant part.

73. A seed coated with the composition of claim 1.

### EXAMPLES

The following examples are provided for illustrative purposes and are not intended to limit the scope of the embodiments as claimed herein. Any variations in the exemplified examples which occur to the skilled artisan are intended to fall within the scope of the present disclosure.

### Materials & Methods

### Bacterial Strains:

Bacillus subtilis var. amyloliquefaciens NRRL B-50349 (Taegro®)

Bacillus subtilis var. amyloliquefaciens ATCC Accession No.: 202152 (TrigoCor)

### Example 1

### Germination of Bacillus Spores at 37° C. In 10 mM Germinant

Bacillus starter cultures were grown in nutrient broth plus yeast extract (Crane et al. 2013), then were transferred 1:1000 to a modified Schaeffer's sporulation medium (Nicholson and Setlow 1990) containing per liter 2.012 g KCl, 0.492 g MgSO₄. 7 H₂0, no NaOH, and 0.0197 g MnCl₂.4H₂0, and grown for 72 h. All cultures were grown in 50 mL aliquots in beveled flasks at 37° C. with 225 RPM shaking. Liquid cultures were spun down at 4° C. for 10 min at 10,000 RCF, washed 10 times in cold deionized water, and resuspended in 5 mL cold deionized water. To remove vegetative cells, cultures were treated with lysozyme (final concentration 50 μg/mL) for 10 min on ice, followed by six rounds of 15 sec sonication on ice, then were washed twice with cold deionized water. Spore preparations were stored in deionized water at 4° C., and prior to use were washed once with cold deionzied water. Unless otherwise noted, spores were resuspended in 25 mM HEPES (Sigma-Aldrich, St. Louis, Mo.) pH 7.41 to produce a final concentration of 10⁸ CFUs/mL. Purified spore cultures contained over 98% phase-bright spores and minimal cell debris, as verified through phase contrast microscopy.

Terbium chloride (1 mM) (Sigma-Aldrich, St. Louis, Mo.) and all germinant solutions were prepared in 25 mM HEPES pH 7.41 and added as noted in individual experiments. 90 μL TrigoCor spores were mixed with 10 μL of each germinant (100 mM, final concentration 10 mM) and incubated in a 96-well plate (Thermo Scientific, Waltham, Mass.) at 37° C. for 1, 10, 30, 60, or 120 min. Samples were prepared so that all incubations finished simultaneously and were analyzed together. For each germinant tested, two spore-germinant mixtures were loaded per incubation time. Germinants tested were D-glucose, D-fructose, KBr, KCl, L-alanine, L-asparagine, L-proline, L-valine, and the germinant combination AGFK (equimolar concentrations of L-asparagine, D-glucose, D-fructose, and KCl), or HEPES as a control. Following addition of 100 μL terbium chloride to all samples, fluorescence was measured as explained below. Assay was repeated twice with comparable results.

At each plate reader measurement time, two 90 μL samples of spores autoclaved for 60 min mixed with 10 μL HEPES, as well as a sample containing a mixture of all germinants and HEPES, were added as additional samples in the plate and mixed with 100 μL terbium chloride. A final sample of pure deionized water was included in the plate as well, and these samples were measured alongside all other samples in the plate. Fluorescence was measured at 545 nm emission and 273 nm excitation according to Yi and Setlow (2010) using a Synergy 4 plate reader (Biotek Instruments Inc., Winooski, Vt.). The samples containing the germinant mixture and deoinized water served as negative controls, and did not produce any significant fluorescence in any of the trials. Percent spore germination for each sample at each time point was calculated by dividing its relative fluorescence units (RFUs) by the corresponding average RFUs of the autoclaved cells, which have released all their dipicolinic acid (DPA) due to lysis (Yang and Ponce 2009) and provided in Table 1. Spore germination results were confirmed using phase contrast microscopy (data not shown).

TABLE 1
Percent germination of TrigoCor spores
at 37° C. in 10 mM germinant. ^{x}

| | Incubation time (min) | | | | |
|---|---|---|---|---|---|
| Germinant | 1 | 10 | 30 | 60 | 120 |
| AGFK ^{y} | 1 ± 0 | 6 ± 1 | 53 ± 3 | 87 ± 1 | 82 ± 11 |
| D-fructose | 1 ± 0 | 1 ± 0 | 1 ± 0 | 1 ± 1 | 1 ± 1 |
| D-glucose | 1 ± 0 | 1 ± 0 | 5 ± 0 | 10 ± 1 | 25 ± 1 |
| Potassium bromide | 1 ± 0 | 1 ± 0 | 1 ± 0 | 1 ± 0 | 1 ± 0 |
| Potassium chloride | 1 ± 0 | 1 ± 0 | 1 ± 0 | 1 ± 0 | 1 ± 0 |
| L-alanine | 1 ± 0 | 4 ± 0 | 20 ± 1 | 23 ± 1 | 21 ± 0 |
| L-asparagine | 1 ± 0 | 1 ± 0 | 1 ± 0 | 1 ± 0 | 1 ± 0 |
| L-proline | 1 ± 0 | 1 ± 0 | 1 ± 0 | 1 ± 0 | 1 ± 0 |
| L-valine | 1 ± 0 | 1 ± 0 | 1 ± 0 | 1 ± 0 | 1 ± 0 |
| Buffer ^{z} | 1 ± 0 | 1 ± 0 | 1 ± 0 | 1 ± 0 | 1 ± 0 |

^{x} Germination was estimated using the terbium chloride assay, and percent germination was calculated by dividing the RFUs of each sample by the average RFUs of samples measured at the same time which had released all their DPA. Numbers shown represent mean percent germination ± standard deviation.
^{y} Equimolar solution of L-asparagine, D-glucose, D-fructose, and potassium chloride.
^{z} HEPES buffer. All germinants and Bacillus spores were prepared in this buffer.

As shown in Table 1, percent germination of spores increases in the presence of AGFK, L-alanine, or D-glucose as compared to control.

### Example 2

### Germination of Bacillus Spores at 37° C. In 10 mM or 100 mM Germinant

The following protocol was performed as per Example 1, with the following modification:

Terbium chloride (1 mM) (Sigma-Aldrich, St. Louis, Mo.) and all germinant solutions were prepared in 25 mM HEPES pH 7.41 and added as noted in individual experiments. 450 μL TrigoCor spores were mixed with 50 μL of each germinant (100 mM and 1 M, final concentrations 10 mM and 100 mM) and incubated at 37° C. Two samples were prepared for each germinant and germinants tested were D-glucose, L-alanine, AGFK, and an equimolar solution of glucose and alanine, or HEPES as a control. After 10, 30, 60, and 120 minutes of incubation, 100 μL was transferred from each tube to a 96-well plate. For an initial measurement of spore germination, two 100 μL samples of spores not mixed with germinants and kept at 4° C. were analyzed alongside the samples which had incubated for 10 minutes. Immediately following each sample time, 100 μL terbium chloride was added to all samples and fluorescence was measured as explained previously. Assay was repeated twice with comparable results.

Percent spore germination for each sample at each time point was calculated by dividing its relative fluorescence units (RFUs) by the corresponding average RFUs of the autoclaved cells, which have released all their dipicolinic acid (DPA) due to lysis (Yang and Ponce 2009) and provided in Table 2. Spore germination results were confirmed using phase contrast microscopy (data not shown).

TABLE 2
Percent germination of TrigoCor spores
at 37° C. in 10 or 100 mM germinant. ^{w}

| | Concentra- | Incubation time (min) | | | |
|---|---|---|---|---|---|
| Germinant | tion (mM) | 10 | 30 | 60 | 120 |
| AGFK ^{x} | 10 | 1 ± 0 | 20 ± 0 | 77 ± 0 | 101 ± 6 |
| AGFK | 100 | 2 ± 0 | 41 ± 1 | 97 ± 3 | 100 ± 4 |
| D-glucose | 10 | 1 ± 0 | 5 ± 0 | 13 ± 1 | 28 ± 2 |
| D-glucose | 100 | 1 ± 0 | 7 ± 1 | 18 ± 2 | 34 ± 1 |
| L-alanine | 10 | 1 ± 0 | 13 ± 1 | 41 ± 1 | 43 ± 1 |
| L-alanine | 100 | 1 ± 0 | 47 ± 1 | 86 ± 3 | 90 ± 3 |
| Alanine-Glucose ^{y} | 10 | 1 ± 0 | 26 ± 2 | 68 ± 1 | 77 ± 5 |
| Alanine-Glucose | 100 | 1 ± 0 | 55 ± 2 | 89 ± 2 | 91 ± 3 |
| Buffer ^{z} | — | 1 ± 0 | 1 ± 0 | 1 ± 0 | 1 ± 0 |

^{w} Germination was estimated using the terbium chloride assay, and percent germination was calculated by dividing the RFUs of each sample by the average RFUs of samples measured at the same time which had released all their DPA. Starting percent germination for all samples was 2 ± 0. Numbers shown represent mean percent germination ± standard deviation.
^{x} Equimolar solution of L-asparagine, D-glucose, D-fructose, and potassium chloride.
^{y} Equimolar solution of L-alanine and D-glucose.
^{z} HEPES buffer. All germinants and Bacillus spores were prepared in this buffer.

As shown in Table 2, percent germination of spores increases in the presence of AGFK, L-alanine, or D-glucose as compared to control. Alanine and glucose combinations worked synergistically and increased the germination enhancement response over either material alone, while AGFK at both concentrations was the superior enhancer. Increasing the concentration of enhancer increases the germination speed.

### Example 3

### Germination of Bacillus Spores at Room Temperature in 10 mM Germinant

Spore preparations were made as in Example 1.

Spore germination was performed in a 96-well plate (USA Scientific, Orlando, Fla.) by mixing 160 μL spores with 40 μL germinant (50 mM, final concentration 10 mM) and incubating at room temperature (24-26° C.) for 6 h, which was previously identified as the time required for the majority of spore germination to occur (data not shown). Three replicates per treatment were analyzed and the assay was performed twice with comparable results. Germinants tested were AGFK and an equimolar solution of L-alanine and D-glucose, and HEPES alone was added as a control. Percent spore germination was estimated using the phase contrast setting of a compound microscope (Carl Zeiss, Oberkochen, Germany) and provided in Table 3. At least 5 fields of view representing a minimum total of 200 spores per replicate were analyzed.

TABLE 3
Percent germination of Bacillus spores
at room temperature in 10 mM germinant. ^{w}

| Germinant | % TrigoCor germination | % Taegro germination |
|---|---|---|
| AGFK ^{x} | 99 ± 1 | 21 ± 6 |
| Alanine-Glucose ^{y} | 39 ± 6 | 1 ± 0 |
| Buffer ^{z} | 2 ± 1 | 0 ± 0 |

^{w} Germination was estimated using phase contrast microscopy after 6 h of incubation. Numbers shown represent mean percent germination ± standard deviation.
^{x} Equimolar solution of L-asparagine, D-glucose, D-fructose, and potassium chloride.
^{y} Equimolar solution of L-alanine and D-glucose.
^{z} HEPES buffer. All germinants and Bacillus spores were prepared in this buffer.

As shown in Table 3, percent germination as seen by microscopy increased in the presence of the germination enhancer AGFK or Alanine+Glucose as compared to a control scenario.

### Example 4

### Germination of Spores on Plant Surfaces Using Germinants

Spore preparations were made as in Example 1.

Spores were resuspended in water and sprayed onto two pots of ‘Norm’ winter wheat as described previously (Crane et al. 2013). Spikes were at late anthesis (Feekes 10.53) at time of application. A second set of two pots were sprayed with sterile deionized water. Wheat spikes were allowed to dry for 4.5 h then 4 spikes per pot were removed for quantification and characterization of Bacillus spore populations as described below. One pot of each treatment was sprayed with approximately 24 mL of either HEPES or AGFK (100 mM). Wheat spikes were allowed to dry for 3 h, then were sprayed with a fine mist of deionized water using a household sprayer (Consolidated Plastics, Stow, Ohio) and covered with a plastic bag (AEP Industries Inc., Peabody, Mass.) to produce a humid environment conducive for spore germination. Plastic bags were removed 24 h later and 5 spikes per pot were removed for quantification and characterization of Bacillus spore populations. Experiment was conducted at room temperature in the laboratory, and was performed twice with comparable results.

Bacillus spore populations from wheat spikes were quantified as described in Jochum et al. (2006), except that spikes were processed individually using 1 mL potassium phosphate buffer amended with 0.1% Triton X-100 per spike. The percentage of Bacillus cells present as dormant spores was estimated via heat treatment as described in Crane et al. (2013) and provided in Table 4.

TABLE 4
Germination of spores on plant surfaces. ^{w}

| | | Bacillus populations | | Dormant spores | |
|---|---|---|---|---|---|
| | | (CFUs/spike) | | (%) ^{x} | |
| Pre- | Germinant | Before | After | Before | After |
| treatment | treatment | treatment | treatment | treatment | treatment |
| Water | AGFK ^{y} | 4 × 10³ ± 5 × 10³ | 1 × 10⁶ ± 4 × 10⁵ | 38 ± 43 | <1 |
| Water | Buffer ^{z} | 5 × 10³ ± 3 × 10³ | 7 × 10⁵ ± 4 × 10⁵ | 24 ± 19 | <1 |
| TrigoCor | AGFK | 4 × 10⁷ ± 9 × 10⁶ | 1 × 10⁸ ± 3 × 10⁷ | 68 ± 13 | 38 ± 11 |
| TrigoCor | Buffer | 2 × 10⁷ ± 3 × 10⁶ | 9 × 10⁷ ± 3 × 10⁷ | 71 ± 2 | <1 |

^{w} AGFK or buffer was applied to wheat spikes pre-treated with either water or TrigoCor spores, then spikes were placed in a humidity chamber for 24 h. Bacillus population numbers and the percentage of CFUs in the dormant spore form were estimated before and after germinant and humidity treatment. Numbers shown represent mean ± standard deviation.
^{x} Dormant spores were estimated by heat treating samples from the dilution series made to quantify population levels. Samples listed as <1 consistently had no detectable CFUs following heat treatment of dilutions that produced approximately 100 CFUs pre-heat treatment.
^{y} Equimolar solution of L-asparagine, D-glucose, D-fructose, and potassium chloride (100 mM) in HEPES buffer.
^{z} HEPES buffer.

As shown in Table 4, populations of Bacillus increase after treatment, and in the presence of AGFK had increased populations as compared to control. AGFK application to wheat spikes pre-treated with TrigoCor consistently produced a greater decline in the percent dormant spores on wheat spikes than did buffer application, indicating that AGFK treatment enhanced spore germination on wheat surfaces.

### LITERATURE CITED

Crane, J. M., D. M. Gibson, R. H. Vaughan, and G. C. Bergstrom. 2013. Iturin levels on wheat spikes linked to biological control of Fusarium head blight by Bacillus amyloliquefaciens. Phytopathology 103 (2):146-155.
Jochum, C. C., L. E. Osborne, and G. Y. Yuen. 2006. Fusarium head blight biological control with Lysobacter enzymogenes strain C3. Biological Control 39 (3):336-344.
Nicholson, W. L., and P. Setlow. 1990. Sporulation, germination and outgrowth. In Molecular Biology Methods for Bacillus, edited by C. R. Harwood and S. M. Cutting: John Wiley & Sons Ltd
Yang, Wan-Wan, and Adrian Ponce. 2009. Rapid endospore viability assay of Clostridium sporogenes spores. International Journal of Food Microbiology 133 (3):213-216.
Yi, Xuan, and Peter Setlow. 2010. Studies of the commitment step in the germination of spores of Bacillus species. Journal of Bacteriology 192 (13):3424-3433.

It will be understood that the Specification and Examples are illustrative of the present embodiments and that other embodiments within the spirit and scope of the claimed embodiments will suggest themselves to those skilled in the art. Although this disclosure has been described in connection with specific forms and embodiments thereof, it would be appreciated that various modifications other than those discussed above may be resorted to without departing from the spirit or scope of the embodiments as defined in the appended claims. For example, equivalents may be substituted for those specifically described, and in certain cases, particular applications of steps may be reversed or interposed all without departing from the spirit or scope of the disclosure as described in the appended claims.

1. A composition comprising:
a. a carrier;
b. one or more microbial spores; and
c. one or more germinants,
wherein the composition is a substantially dry composition.

2. The composition of claim 1, wherein the germinant is selected from the group consisting of lactate, lactose, bicarbonate, fructose, glucose, mannose, galactose, alanine, asparagine, cysteine, glutamine, norvatine, serine, threonine, valine, glycine, inosine, taurocholate, and combinations thereof.

3. The composition of claim 1, wherein the germinant is a combination of L-asparagine, glucose, fructose, and potassium ion (AGFK).

4. The composition of claim 1, wherein the one or more microbial spores is one or more bacterial spores.

5. The composition of claim 4, wherein the one or more bacterial spores are one or more Bacillus spores.

6. The composition of claim 5, wherein the one or more Bacillus spores are selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.

7. The composition of claim 1, wherein the composition further comprises one or more agriculturally beneficial ingredients.

8. The composition of claim 7, wherein the one or more agriculturally beneficial ingredients is one or more biologically active ingredients.

9. The composition of claim 8, wherein the one or more biologically active ingredients are selected from the group consisting of one or more plant signal molecules, one or more beneficial microorganisms, and combinations thereof.

10. A method for treating a plant or plant part comprising contacting a plant or plant part with
a. one or more microbial spores; and
b. one or more germinants.

11. The method of claim 10, wherein the contacting comprises foliarly applying to a plant or plant part one or more microbial spores and one or more germinants.

12. The method of claim 10, wherein the germinant is selected from the group consisting of lactate, lactose, bicarbonate, fructose, glucose, mannose, galactose, alanine, asparagine, cysteine, glutamine, norvatine, serine, threonine, valine, glycine, inosine, taurocholate, and combinations thereof.

13. The method of claim 10, wherein the germinant is a combination of L-asparagine, glucose, fructose, and potassium ion (AGFK).

14. The method of claim 10, wherein the one or more microbial spores is one or more bacterial spores.

15. The method of claim 14, wherein the one or more bacterial spores are one or more Bacillus spores.

16. The method of claim 15, wherein the one or more Bacillus spores are selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.

17. The method of claim 10, wherein the method further comprises applying to the plant or plant part one or more agriculturally beneficial ingredients.

18. A method for inducing the germination of a microbial spore comprising foliarly applying one or more microbial spores and one or more germinants to a plant or plant part, wherein upon foliar application of the one or more microbial spores and the one or more germinants to a plant or plant part, the one or more microbial spores exhibit increased germination on the plant or plant part in the presence of the one or more germinants compared to the foliar application of one or more microbial spores on a plant or plant part without the one or more germinants.

19. The method of claim 18, wherein the germinant is a combination of L-asparagine, D-glucose, D-fructose, and potassium ion (AGFK).

20. The method of claim 18, wherein the one or more microbial spores are Bacillus spores selected from the group consisting of Bacillus pumilus isolate AQ717 having the deposit accession number NRRL B-21662, Bacillus pumilus having the deposit accession number NRRL B-30087, Bacillus sp. isolate AQ175 having the deposit accession number ATCC 55608, Bacillus sp. isolate AQ177 having the deposit accession number ATCC 55609, Bacillus subtilis isolate AQ713 having the deposit accession number NRRL B-21661, Bacillus subtilis isolate AQ743 having the deposit accession number NRRL B-21665, Bacillus amyloliquefaciens having the deposit accession number NRRL B-50304, Bacillus amyloliquefaciens having the deposit accession number NRRL B-50349, Bacillus amyloliquefaciens TJ1000 having the deposit accession number ATCC BAA-390, Bacillus thuringiensis isolate AQ52 having the deposit accession number NRRL B-21619, Bacillus subtilis var. amyloliquefaciens the deposit accession number ATCC 202152, and combinations thereof.
